(19) United States
(12) Patent Application Publication
(10) Pub. No.: US 2025/0082611 A1
(43) Pub. Date: Mar. 13, 2025

(54) DOSING REGIMEN FOR AN NLRP3 INHIBITOR IN THE TREATMENT OF OSTEOARTHRITIS

(71) Applicant: Novartis AG, Basel (CH)

(72) Inventors: Laura COLEMAN, Boston, MA (US); Christopher FARADY, Basel (CH); Ewa GATLIK, Basel (CH); Matthias SCHIEKER, Munich (DE)

(21) Appl. No.: 18/580,781

(22) PCT Filed: Jul. 20, 2022
(86) PCT No.: PCT/IB2022/056695

§ 371 Date: Jan. 19, 2024

Related U.S. Application Data
(60) Provisional application No. 63/224,890, filed on Jul. 23, 2021.

(51) Int. Cl. A61K 31/426; A61P 19/02

(52) U.S. Cl. CPC A61K 31/426; A61P 19/02

(57) ABSTRACT

The present disclosure relates to the field of pharmacy, particularly to an NLRP3 inhibitor for use in the treatment of osteoarthritis. The disclosure also relates to an NLRP3 inhibitor or a pharmaceutical combination comprising an NLRP3 inhibitor, and at least one further therapeutic agent, for use in the treatment of osteoarthritis; to a method for the treatment of osteoarthritis that involves administering an NLRP3 inhibitor or the combination; and to the use of an NLRP3 inhibitor or the combination for the manufacture of a medicament for the treatment of osteoarthritis.

### TECHNICAL FIELD

The present disclosure relates to the field of pharmacy, particularly to an NLRP3 inhibitor for use in the treatment of osteoarthritis. The disclosure also relates to an NLRP3 inhibitor or a pharmaceutical combination comprising an NLRP3 inhibitor, and at least one further therapeutic agent, for use in the treatment of osteoarthritis; to a method for the treatment of osteoarthritis that involves administering an NLRP3 inhibitor or the combination; and to the use of an NLRP3 inhibitor or the combination for the manufacture of a medicament for the treatment of osteoarthritis.

### BACKGROUND OF THE INVENTION

Osteoarthritis (OA), the most common joint disorder in the world, is a serious, chronic progressive joint disease with no known cure that has been associated with an increased risk of premature mortality (Osteoarthritis Research Society International 2016, Paper submitted to U.S. Food and Drug Administration; 1 December; Kluzek et al 2015, Ann Rheum Dis. 75 (10): 1749-56).

Clinically, OA is associated with joint pain, swelling and stiffness that can lead to activity limitations, sleep interruption, fatigue, depression, anxiety, and ultimately loss of independence and reduced quality of life (Osteoarthritis Research Society International 2016).

According to clinical practice guidelines (Bannuru et al 2019, Cartilage; 27:1578-1589; Kolasinski et al 2020, Arthritis and Rheumatology; 72:220-33), nonsurgical treatment of OA comprises both pharmacologic and nonpharmacologic modalities (such as patient education, referral to a physiotherapist, exercise, weight reduction, walking aids, knee braces, and footwear). All presently available pharmacologic therapies for OA provide symptomatic relief by transiently reducing pain but have not been shown to delay the structural damage associated with the progression of OA. In addition, long-term use of these therapies may be associated with serious adverse effects, including fall-related bone fractures, substance dependence and/or abuse in patients receiving opioids, cardiovascular risk, and upper gastrointestinal bleeding in OA patients receiving nonsteroidal anti-inflammatory drugs (Fernandes et al, 2013, Ann Rheum Dis. 72 (7): 1125-35; McAlindon et al, 2014, Osteoarthritis Cartilage; 22 (3): 363-88; Nissen et al, 2016, NEJM 375:2519-29; Chan et al 2017, Lancet 389:2375-82; Soloman et al 2017, Am. J. Med.; 130:1415-22; Kolasinski et al 2020).

Total knee replacement surgery (TKR) is considered when adequate attempts of symptomatic pharmacologic therapies fail (Bannuru et al 2019; Kolasinski et al 2020). However, not all patients are satisfied with the result, or benefit from joint replacement surgery. As longevity increases and OA prevalence rises (even at younger ages), the ever increasing number of joint replacement surgeries causes a growing public health burden (Losina and Katz et al 2012, Arthritis Rheum. 64 (2): 339-41).

Pro-inflammatory cytokines such as interleukin-1β (IL-1β) are critical mediators of the disturbed metabolism and enhanced catabolismof joint tissue involved in OA (Fraenkel et al 1998, J Rheumatol., 1820-6), making anti-inflammatory therapy an attractive strategy to counteract OA. These inflammatory mediators induce downregulation of anabolic events, i.e. cartilage matrix production by chondrocytes and production of degrading enzymes (MMPs, ADAMTS) by chondrocytes and synovial cells, which cause the breakdown and loss of the cartilage matrix (van den Bosch 2019, Clin Exp Immunol. 153-166).

Through the production of IL-1β and IL-18, the NLRP3 inflammasome has been implicated as a major driver of inflammation associated with many chronic inflammatory diseases. Mechanistically, NLRP3 senses a diverse range of danger signals, and responds by forming an inflammasome protein complex that drives an inflammatory response. NLRP3 inhibitors have been shown to block IL-1β secretion, IL-18 secretion and pyroptotic cell death in response to a wide variety of NLRP3-dependent danger signals in vitro and in mechanistic mouse models in vivo.

There is an unmet need for a disease-modifying osteoarthritis drug (DMOAD) that can slow or halt OA disease progression by inhibiting structural deterioration and improving symptoms. Although many putative agents have been investigated, no pharmaceutical agent has been approved for clinical use as a DMOAD (Tonge et al 2014, Osteoarthritis Cartilage.; 22 (5): 609-21; Karsdal et al 2016, Osteoarthritis and Cartilage, 24:2013-21; Oo et al 2018, Expert Opin Emerg Drugs Dec; 23 (4): 331-347; Alcaraz et al 2019, Biochem Pharmacol. 165:4-16).

### SUMMARY OF THE INVENTION

Provided herein are NLRP3 inhibitors which may be used to prevent or reduce the NLRP3 inflammasome response and thus address the unmet medical need in OA. For example, an NLRP3 inhibitor disclosed herein can be developed as an OA drug to reduce pain, slow joint damage and improve function in adults with symptomatic OA by addressing the inflammatory aspect of the disease and delaying/preventing progression to end-stage OA.

In one aspect, the present invention relates to methods for treating OA, e.g. knee OA, hand OA, hip OA, spinal OA, foot and ankle OA, by administering therapeutically effective amounts of an NLRP3 inhibitor, in particular Compound I or a pharmaceutically acceptable salt thereof, to a subject. Described herein are also NLRP3 inhibitors, in particular Compound I or a pharmaceutically acceptable salt thereof, for use in treating OA, e.g. knee OA, hand OA, hip OA, spinal OA, foot and ankle OA.

Further provided herein are specific dose regimens for the methods or use of an NLRP3 inhibitor in treating OA, in particular Compound I or a pharmaceutically acceptable salt thereof, as described herein.

Additionally described herein are pharmaceutical combinations comprising a) Compound I or a pharmaceutically acceptable salt thereof, and b) at least one further therapeutic agent, optionally in the presence of a pharmaceutically acceptable carrier, in the treatment of OA and pharmaceutical compositions or kits comprising them.

In certain embodiments, Compound I is Compound IA.

Further features and advantages of the described methods and uses will become apparent from the following detailed description.

### DESCRIPTION OF THE DRAWINGS

FIG. 1: A schematic overview of the treatment protocol as detailed in Example 1.

FIG. 2: A schematic overview of the study design of the first-in-human (FIH) study as detailed in Example 2.

### DETAILED DESCRIPTION OF THE INVENTION

### Definitions

In order that the present document may be more readily understood, certain terms are first defined. Additional definitions are set forth throughout this document.

All patents, published patent applications, publications, references and other material referred to herein are incorporated by reference herein in their entirety for the described purpose.

As used herein, the articles “a”, “an”, and “the” in both the description and claims are to be construed to cover both the singular and the plural, unless otherwise indicated herein or clearly contradicted by context. The terms “comprising”, “having”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “or” is used herein to mean, and is used interchangeably with, the term “and/or”, unless context clearly indicates otherwise.

The term “about” or “approximate” in relation to a reference numerical value and its grammatical equivalents as used herein can include the numerical value itself and a range of values plus or minus 20% (preferably ±15%, more preferably ±10%, even more preferably ±5%) from that numerical value. For example, the amount “about 10” includes 10 and any amounts from 8 to 12 or from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical value described throughout can be “about” that numerical value even without specifically mentioning the term “about.”

As used herein, the term “baseline” refers to a subject's state or the degree of a condition, e.g., a disease, or one or more parameters associated with the state of a patient, observed before treatment, e.g., before administration of a compound, e.g., before administration of Compound I, or a pharmaceutically acceptable salt thereof, optionally in combination with at least one further therapeutic agent, according to the described methods and uses.

As used herein, the term “administering” in relation to a compound, e.g., Compound I optionally in combination with at least one further therapeutic agent, is used to refer to delivery of that compound by any route of delivery. Such delivery may be, for example, an intravenous administration or oral administration. Such delivery may also be, for example, a subcutaneous administration.

As used herein, the word “substantially” does not exclude “completely,” e.g., a composition which is “substantially free” from Y may be completely free from Y. Where necessary, the word “substantially” may be omitted from the definition.

As used herein, the term “pharmaceutically acceptable” means a nontoxic material that does not substantially interfere with the effectiveness of the biological activity of the active ingredient(s).

As used herein, the term “patient” is used interchangeably with the term “subject” and includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. In a specific embodiment, the compositions, methods, and uses described herein are in reference to a human patient or subject.

As used herein, a subject is “in need of” a treatment if such subject who is afflicted with the condition (i.e., disease, disorder, or syndrome) of interest and who would benefit biologically, medically, or in quality of life from such treatment.

The term “treatment,” “treating,” or “treat” is herein defined as therapeutic measures for the reduction or amelioration of the progression, severity and/or duration of an undesired physiological change or disorder (e.g. OA such knee OA), or the amelioration of one or more symptoms (preferably, one or more discernible symptoms) of the disorder resulting from the administration of one or more therapeutic agents. In other embodiments the terms “treatment,” “treating,” or “treat” refer to the reduction or stabilization of the progression of a disorder, such as OA, either physically by, e.g., reduction or stabilization of a discernible symptom, physiologically by, e.g., reduction or stabilization of a physical parameter, or both. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and reversal (whether partial or total), whether detectable or undetectable.

For example, “treating OA such as knee OA, hand OA, hip OA, spinal OA, foot and ankle OA” may refer to ameliorating, alleviating or modulating at least one of the symptoms or pathological features associated with OA; e.g. reduce pain, slow joint damage and improve function; e.g., may refer to slowing progression, reducing or stopping at least one of the symptoms or pathological features associated with OA. It may also refer to preventing or delaying one or more of the described symptoms, e.g., slow the progress of, halt, or reverse disease, condition, disorder, manifestation or syndrome progression and improve clinical outcomes.

Also “treating” may refer to slow the progress of, halt, or reverse disease, condition, disorder, manifestation or syndrome progression and improve clinical outcomes, e.g., moving from a higher number to a lower number on a 5-point scale of disease-associated clinical signs and symptoms as follows:

| Scale Number | Clinical signs and Symptoms |
|---|---|
| 0 | Absent |
| 1 | Minimal |
| 2 | Mild |
| 3 | Moderate |
| 4 | Severe |

As used herein, term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

As used herein, the term “NLRP3 inhibitor” is a compound that inhibits the ability of NLRP3 to induce the production of IL-1β and/or IL-18 by directly binding to NLRP3, or by inactivating, destabilizing, altering distribution, of NLRP3 or otherwise. Typically, an NLRP3 inhibitor has an IC50 of <1 UM of IL-1β secretion in the hTHP-1 assay containing 2% fetal bovine serum defined herein.

Preferably, the NLRP3 inhibitor is a compound of Compound I, Compound IA, or Compound IB, or a pharmaceutically acceptable salt thereof. More preferably, the NLRP3 inhibitor is Compound IA, or a pharmaceutically acceptable salt thereof.

As used herein, “Compound of formula I,” or “Compound I,” are used interchangeably and mean a compound that has the structure shown below, and can be synthesized using procedures known in the art and described in WO2019/023147, incorporated by reference in its entirety.

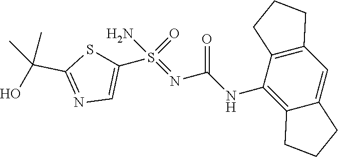 [SMILES: CC(C)(O)c1ncc(S(N)(=O)=NC(=O)Nc2c3c(cc4c2CCC4)CCC3)s1]

Compound I, Compound IA (i.e., (R)-N′-((1,2,3,5,6,7-hexahydro-s-indacen-4-yl) carbamoyl)-2-(2-hydroxypropan-2-yl) thiazole-5-sulfonimidamide) or Compound IB (i.e., (S)-N′-((1,2,3,5,6,7-hexahydro-s-indacen-4-yl) carbamoyl)-2-(2-hydroxypropan-2-yl) thiazole-5-sulfonimidamide) may be used in crystalline or amorphous form, as a solvate, e.g., a hydrate, or an unsolvated form.

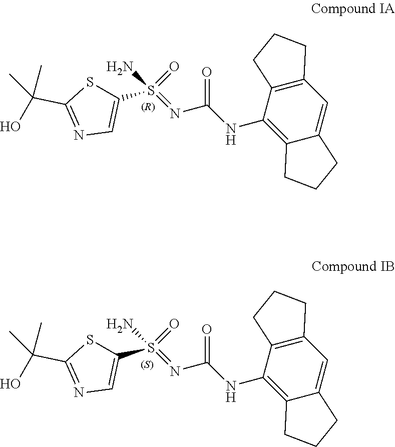

### Tautomers:

The scope of the compounds disclosed herein includes tautomeric form of the compounds. Thus, by way of example, a compound that is represented as containing the moiety

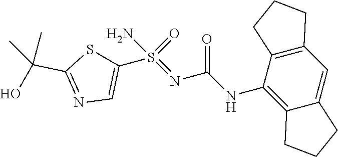 [SMILES: CC(C)(O)c1ncc(S(N)(=O)=NC(=O)Nc2c3c(cc4c2CCC4)CCC3)s1]

is also intended to include the tautomeric form containing the moiety

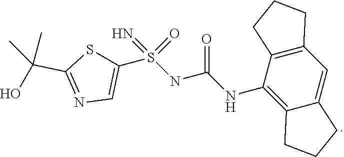 [SMILES: CC(C)(O)c1ncc(S(=N)(=O)NC(=O)Nc2c3c(cc4c2CCC4)CCC3)s1]

### Stereoisomers:

Non-limiting exemplified compounds of the formulae described herein include a stereogenic sulfur atom. This disclosure provides examples of stereoisomer mixtures (e.g. racemic mixture of enantiomers). This disclosure also describes and exemplifies methods for separating individual components of said stereoisomer mixtures (e.g. resolving the enantiomers of a racemic mixture). Compound I, for example, represents each of a non-racemic mixture of Compound IA and Compound IB, a racemic mixture of Compound IA and Compound IB; Compound IA in enantiomerically pure form; or Compound IB in enantiomerically pure form. As used herein, “Compound I” is also intended to include enantiomeric excesses of either Compound IA or Compound IB. For example, Compound IA may be present in an enantiomeric excess of about 90%, about 91%, about 92%, about 93%, about 94%, about 95%, about 96%, about 97%, about 98%, about 99%, or about 99.5%. Alternatively, Compound IB may be present in an enantiomeric excess of about 90%, about 91%, about 92%, about 93%, about 94%, about 95%, about 96%, about 97%, about 98%, about 99%, or about 99.5%.

Any chemical formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the disclosure include, for example, isotopes of hydrogen, carbon, nitrogen, and oxygen, such as ³H, ¹¹C, ¹³C, ¹⁴C, and ¹⁵N. Accordingly, it should be understood that methods of the present invention can or may involve compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds can generally be prepared by conventional techniques known to those skilled in the art, e.g., using an appropriate isotopically-labeled reagents in place of the non-labeled reagent previously employed.

The present invention encompasses embodiments that include all pharmaceutically acceptable salts of the compounds useful according to the invention provided herein. As used herein, “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts of the parent compound formed, for example, from non-toxic inorganic or organic acids. The pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. For example, preferred pharmaceutically acceptable salts include, but are not limited to, mineral or organic acid salts of basic residues such as amines. For example, the salt can be a hydrochloride salt.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Unless otherwise indicated, as used here, the “dose” or amount of NLRP3 inhibitor, e.g. Compound I, or a pharmaceutically acceptable salt thereof, refers to the amount of the free base or free acid form of the compound. For salt forms of the NLRP3 inhibitor, the actual amount will be adjusted based on the salt form used.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease, condition, disorder, or syndrome or related symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week, and also include less frequent administration, e.g., as described herein. The skilled artisan will appreciate that certain factors may influence the dosage and timing required to effectively treat a subject, including but not limited to the severity of the disease, condition, disorder, or syndrome, previous treatments, the general health and/or age of the subject, and other concurrent diseases, conditions, disorders, or syndromes. Moreover, treatment of a subject with a therapeutically effective amount of the therapeutic compounds described herein can include a single treatment or a series of treatments.

As used herein, the term “therapeutically effective amount” of the compound described herein refers to an amount of the compound that will elicit the biological or medical response of a subject, for example, ameliorate symptoms, alleviate conditions, slow or delay disease progression, or prevent a disease, condition, disorder, manifestation or syndrome, etc. In one non-limiting embodiment, the term “a therapeutically effective amount” refers to the amount of the compound described herein that, when administered to a subject, is effective to at least partially ameliorating, alleviating or modulating at least one of the symptoms or pathological features associated with OA; e.g. reduce pain, slow joint damage and improve function; e.g., may refer to slowing progression, reducing or stopping at least one of the symptoms or pathological features associated with OA. It may also refer to preventing or delaying one or more of the described symptoms, e.g., slow the progress of, halt, or reverse disease, condition, disorder, manifestation or syndrome progression and improve clinical outcomes.

As herein defined, “combination” refers to either a fixed combination in one unit dosage form (e.g., capsule, tablet, sachet or vial), free (i.e., non-fixed) combination, or a kit of parts for the combined administration where Compound I, or a pharmaceutically acceptable salt thereof, and the one or more additional therapeutic agents may be administered independently at the same time or separately within time intervals, especially where these time intervals allow that the combination partners show a cooperative, e.g., synergistic effect.

The terms “co-administration” or “combined administration” or the like as utilized herein are meant to encompass administration of an additional therapeutic agent to a single subject in need thereof (e.g., a subject), and the additional therapeutic agent are intended to include treatment regimens in which Compound I and additional therapeutic agent are not necessarily administered by the same route of administration and/or at the same time. Each of the components of the presently described combination may be administered simultaneously or sequentially and in any order. Co-administration comprises simultaneous, sequential, overlapping, interval, and/or continuous administrations and any combination thereof.

The term “pharmaceutical combination” as used herein means a pharmaceutical composition that results from the combining (e.g., mixing) of more than one active ingredient and includes both fixed and free combinations of the active ingredients.

The term “fixed combination” means that the active ingredients are administered to a subject simultaneously in the form of a single entity or dosage.

The term “free combination” (non-fixed combination) means that the active ingredients as defined herein are administered to a subject as separate entities either simultaneously, concurrently or sequentially with no specific time limits, and in any order, wherein such administration provides therapeutically effective levels of the compounds in the subject's body. In particular, reference to the combination comprising a) Compound I and b) at least one additional therapeutic agent as used herein (e.g., in any of the embodiments or in any of the claims herein), refers to a “non-fixed combination” and may be administered independently at the same time or separately within time intervals.

By “simultaneous administration”, it is meant that the active ingredients as defined herein, are administered on the same day. The active ingredients can be administered at the same time (for fixed or free combinations), or one at a time (for free combinations). The term “sequential administration”, may mean that during a period of two or more days of continuous co-administration only one of active ingredients as herein defined, is administered on any given day.

By “overlapping administration”, it is meant that during a period of two or more days of continuous co-administration, there is at least one day of simultaneous administration and at least one day when only one of active ingredients as herein defined, is administered.

By “continuous administration”, it is meant a period of co-administration without any void day. The continuous administration may be simultaneous, sequential, or overlapping, as described above.

The term “dose” refers to a specified amount of a drug administered at one time. The dose could, for example, be declared on a product package or in a product information leaflet.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP3 molecules, isoforms, precursors, mutants, variants, derivatives, splice variants, alleles, different species, and active fragments thereof.

The phrase “means for administering” is used to indicate any available implement for systemically administering a drug to a patient, including, but not limited to, a dropper, a pre-filled syringe, a vial and syringe, an injection pen, an autoinjector, an i.v. drip and bag, a pump, etc. With such items, a patient may self-administer the drug (i.e., administer the drug on their own behalf), a caregiver may administer the drug to the patient, or a physician or other medical professional may administer the drug.

### Methods of Treatment

The present invention provides a method of treating osteoarthritis comprising administering an NLRP3 inhibitor to a subject. In some embodiments, the osteoarthritis is knee, hand, hip, or spine osteoarthritis.

Treatment with an NLRP3 inhibitor compound according to one of the dosing regimens described herein is expected to slow or halt the progress of osteoarthritis and reduce or eliminate symptoms associated with osteoarthritis as compared to treatment with placebo. In one non-limiting example, treatment may decrease pain as measured by the Knee Injury and Osteoarthritis Outcome Score (KOOS) or the Western Ontario and McMaster Universities Osteoarthritis Index (WOMAC) score based on change from baseline. In another embodiment, treatment may reduce stiffness associated with osteoarthritis. In another embodiment, treatment may result in the reduction of the inflammation level of the osteoarthritis affected joint as determined by change from baseline in synovitis activity level measured from K^{trans} by dynamic contrast-enhanced (DCE)-MRI. In another non-limiting example, treatment according to one of the presently described dosing regimens may improve or maintain (e.g., prevent further decrease) the function in the affected joint. In another non-limiting example, treatment according to one of the presently described dosing regimens may prolong the survival of the joint affected with osteoarthritis and/or increase the subject's quality of life. In yet another non-limiting example, treatment according to a dosage regimen of the present invention may prevent or delay the need for joint replacement surgery. Treatment according to the dosing regimens described below may continue until such time as the subject no longer receives a therapeutic benefit.

The NLRP3 inhibitor may be administered according to any known administration method. In certain preferred embodiments, the NLRP3 inhibitor is administered via oral administration, e.g., as a tablet. Other possible routes of administration include, e.g., intradermal, intramuscular, intravenous, and intra-articular. The NLRP3 inhibitor may also be administered according to any known means for administering a therapeutic to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

### Doses and Dosing Regimens

The methods of treatment of the present invention comprise administering an NLRP3 inhibitor according to a dosing regimen. In an embodiment 1, the dosing regimen comprises administering an NLRP3 inhibitor at a total daily dose of about 10 mg to about 100 mg in a single dose or divided doses to a subject. In an embodiment 2, the dosing regimen comprises administering an NLRP3 inhibitor at a total daily dose of about 20 mg to about 50 mg in a single dose or divided doses to a subject. In an embodiment 3, the dosing regimen comprises administering an NLRP3 inhibitor at a total daily dose of about 20 mg in a single dose or divided doses to a subject. In an embodiment 4, the dosing regimen comprises administering an NLRP3 inhibitor at a total daily dose of about 50 mg in a single dose or divided doses to a subject. In an embodiment 5, the dosing regimen comprises administering an NLRP3 inhibitor at about 10 mg twice daily to a subject. In an embodiment 6, the dosing regimen comprises administering an NLRP3 inhibitor at about 25 mg twice daily to a subject. In an embodiment 7, the dosing regimen comprises administering an NLRP3 inhibitor at about 10 mg twice daily to a subject for about 14 consecutive days. In an embodiment 8, the dosing regimen comprises administering an NLRP3 inhibitor at about 25 mg twice daily to a subject for about 70 consecutive days. In another embodiment 9, the doses are administered to a subject during or after consuming food. In an embodiment 10, the time interval between the administration of two subsequent doses is about 10-14 hours. In a preferred embodiment 11, the methods of treatment relate to the treatment of knee osteoarthritis. In another preferred embodiment 12, the subject in the methods of treatment is a human subject. In another embodiment 13, the administration of the NLRP3 inhibitor decreases pain in the osteoarthritis affected joint as determined by KOOS score based on change from baseline. In another embodiment 14, the administration of the NLRP3 inhibitor reduces the inflammation level of the osteoarthritis affected joint as determined by change from baseline in synovitis activity level measured from K^{trans} by dynamic contrast-enhanced (DCE)-MRI. In another embodiment 15, the level of serum high sensitivity C-Reactive Protein decreases in a subject by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% as determined by change from baseline. In another embodiment 16, the level of IL-1β or IL-18 decreases in a subject by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% as determined by change from baseline. In another embodiment 17, the subject does not exhibit any skin rash. In another embodiment 18, the NLRP3 inhibitor is administered to the subject orally. In another embodiment 19, the NLRP3 inhibitor is comprised in a tablet formulation. In another embodiment 20, at least one further therapeutic agent is administered. In another embodiment 21, the NLRP3 inhibitor is Compound I, or a pharmaceutically acceptable salt thereof:

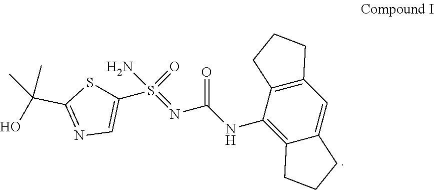 [SMILES: CC(C)(O)c1ncc(S(N)(=O)=NC(=O)Nc2c3c(cc4c2CCC4)CCC3)s1]

In another embodiment 22, the NLRP3 inhibitor is Compound IA, or a pharmaceutically acceptable salt thereof. In another embodiment 23, Compound IA has an enantiomeric excess of at least 90%, at least 91%, at least 92%, at least 93%, at least 94%, at least 95%, at least 96%, at least 97%, at least 98%, at least 99%, or at least 99.5%. In another embodiment 24, the NLRP3 inhibitor is Compound IB, or a pharmaceutically acceptable salt thereof. In another embodiment 25, Compound IB has an enantiomeric excess of at least 90%, at least 91%, at least 92%, at least 93%, at least 94%, at least 95%, at least 96%, at least 97%, at least 98%, at least 99%, or at least 99.5%.

As is understood by a person skilled in the art, the above embodiments of the present invention may be combined with each other.

Further embodiments of the present invention (embodiments 26.1 to 26.30):
26.1 An NLRP3 inhibitor for use in the treatment of osteoarthritis or the use of an NLRP3 inhibitor in the manufacture of a medicine for the treatment of osteoarthritis wherein the NLRP3 inhibitor is administered to a subject at a total daily dose of about 10 mg to about 100 mg in a single dose or divided doses.
26.2 The NLRP3 inhibitor for use according to embodiment 26.1, wherein the NLRP3 inhibitor is administered to a subject at a total daily dose of about 20 mg to about 50 mg in a single dose or divided doses.
26.3 The NLRP3 inhibitor for use according to embodiment 26.1 or 26.2, wherein the NLRP3 inhibitor is administered to a subject at a total daily dose of about 20 mg in a single dose or divided doses.
26.4 The NLRP3 inhibitor for use according to any one of embodiments 26.1 to 26.3, wherein the NLRP3 inhibitor is administered to a subject at a total daily dose of about 50 mg in a single dose or divided doses.
26.5 The NLRP3 inhibitor for use according to any one of embodiments 26.1 to 26.4, wherein the NLRP3 inhibitor is administered to a subject at a dose of about 10 mg twice daily.
26.6 The NLRP3 inhibitor for use according to any one of embodiments 26.1 to 26.5, wherein the NLRP3 inhibitor is administered to a subject at a dose of about 10 mg twice daily for about 14 consecutive days.
26.7 The NLRP3 inhibitor for use according to any one of embodiments 26.1 to 26.6, wherein the NLRP3 inhibitor is administered to a subject at a dose of about 25 mg twice daily.
26.8 The NLRP3 inhibitor for use according to any one of embodiments 26.1 to 26.7, wherein the NLRP3 inhibitor is administered to a human subject at a dose of about 25 mg twice daily for about 70 consecutive days.
26.9 The NLRP3 inhibitor for use according to any one of embodiments 26.1-26.8, wherein the NLRP3 inhibitor is administered to a subject during or after consuming food.
26.10 The NLRP3 inhibitor for use according to any one of embodiments 26.1-26.9, wherein there is about a 10-14 hour time interval between the administration of two subsequent doses of the NLRP3 inhibitor to a subject.
26.11 The NLRP3 inhibitor for use according to any one of embodiments 26.1-26.10, wherein said osteoarthritis is knee osteoarthritis.
26.12 The NLRP3 inhibitor for use according to any one of embodiments 26.1-26.11, wherein administration of the NLRP3 inhibitor decreases pain in the osteoarthritis affected joint as determined by KOOS score based on change from baseline.
26.13 The NLRP3 inhibitor for use according to any one of embodiments 26.1-26.12, wherein administration of the NLRP3 inhibitor reduces the inflammation level of the osteoarthritis affected joint as determined by change from baseline in synovitis activity level measured from K^{trans} by dynamic contrast-enhanced (DCE)-MRI.
26.14 The NLRP3 inhibitor for use according to any one of embodiments 26.1-26.13, wherein the level of serum high sensitivity C-Reactive Protein decreases in a subject by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% as determined by change from baseline.
26.15 The NLRP3 inhibitor for use according to any one of embodiments 26.1-26.14, wherein the level of IL-1β or IL-18 decreases in a subject by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% as determined by change from baseline.
26.16 The NLRP3 inhibitor for use according to any one of embodiments 26.1-26.15, wherein the subject does not exhibit any skin rash.
26.17 The NLRP3 inhibitor for use according to any one of embodiments 26.1-26.16, wherein the NLRP3 inhibitor is administered to the subject orally.
26.18 The NLRP3 inhibitor for use according to any one of embodiments 26.1-26.17, wherein the NLRP3 inhibitor is comprised in a tablet formulation.
26.19 The NLRP3 inhibitor for use according to any one of embodiments 26.1-26.18, comprising administering at least one further therapeutic agent.
26.20 The NLRP3 inhibitor for use according to any one of embodiments 26.1-26.19, wherein the NLRP3 inhibitor is Compound I, or a pharmaceutically acceptable salt thereof:

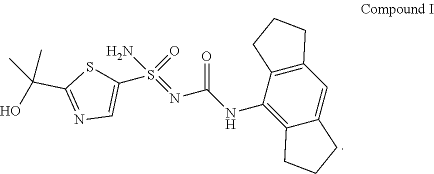 [SMILES: CC(C)(O)c1ncc(S(N)(=O)=NC(=O)Nc2c3c(cc4c2CCC4)CCC3)s1]

26.21 The NLRP3 inhibitor for use according to embodiment 26.20, wherein the NLRP3 inhibitor is Compound IA, or a pharmaceutically acceptable salt thereof.
26.22 The NLRP3 inhibitor for use according to embodiment 26.21, wherein Compound IA has an enantiomeric excess of at least 90%, at least 91%, at least 92%, at least 93%, at least 94%, at least 95%, at least 96%, at least 97%, at least 98%, at least 99%, or at least 99.5%.
26.23 The NLRP3 inhibitor for use according to embodiment 26.20, wherein the NLRP3 inhibitor is Compound IB, or a pharmaceutically acceptable salt thereof.
26.24 The NLRP3 inhibitor for use according to embodiment 26.23, wherein Compound IB has an enantiomeric excess of at least 90%, at least 91%, at least 92%, at least 93%, at least 94%, at least 95%, at least 96%, at least 97%, at least 98%, at least 99%, or at least 99.5%.
26.25 The NLRP3 inhibitor for use according to any one of embodiments 26.1-26.24, wherein the subject is a human subject.
26.26 A pharmaceutical composition comprising an NLRP3 inhibitor of embodiments 26.20 to 26.24, for use according to any one of embodiments 26.1 to 26.25.

Various embodiments of the methods and uses described herein are included below and elsewhere in the document. It will be recognized that features specified in each embodiment may be combined with other specified features to provide further embodiments.

It is taught herein that the below embodiments relate to the use of any NLRP3 inhibitor, and is not limited to Compound I. Preferably, Compound I of the below embodiments is Compound IA (i.e. the R enantiomer) in an enantiomeric excess of at least 90%, at least 91%, at least 92%, at least 93%, at least 94%, at least 95%, at least 96%, at least 97%, at least 98%, at least 99%, or at least 99.5%. Preferably, Compound IA is in enantiomeric excess of at least 90%. More preferably, Compound IA is in enantiomeric excess of at least 95%.

In some embodiments, provided herein is a pharmaceutical composition comprising Compound I or a pharmaceutically acceptable salt thereof, and at least one pharmaceutically acceptable excipient. In particular embodiments, the pharmaceutical composition is a tablet. In yet particular embodiments, the pharmaceutical composition is administered as a whole or crushed tablet. In some embodiments, the pharmaceutical composition includes about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, or about 100 mg in each unit dose.

Provided herein is a pharmaceutical composition comprising Compound I, or a pharmaceutically acceptable salt thereof, for use in any of the embodiments described herein.

In any of embodiments described herein, Compound I, or a pharmaceutically acceptable salt thereof, is administered to a subject in need thereof orally. In some embodiments, Compound I, or a pharmaceutically acceptable salt thereof, is in the form of a tablet that is administered either whole or subdivided, i.e., crushed prior to administration. In particular embodiments, for example when patients are unable to swallow, Compound I may be administered via a nasogastric tube.

### Synthesis of Compound I

Compounds I, IA and IB were synthesized in accordance with the synthesis defined in WO2019/023147 for examples 4, 5 and 6, and as detailed below. The compounds, however, may be assembled in various ways, building up the final molecules using related reaction procedures in a modular fashion which allows for different reaction orders and/or different reagents.

The progress of reactions was often monitored by TLC or LC-MS. The identity of the products was often confirmed by LC-MS. The LC-MS was recorded using the following method:

Method A: Shim-pack XR-ODS, C18, 3×50 mm, 2.5 um column, 1.0 uL injection, 1.5 mL/min flow rate, 90-900 amu scan range, 190-400 nm UV range, 5-100% (1.1 min), 100% (0.6 min) gradient with ACN (0.05% TFA) and water (0.05% TFA), 2 minute total run time.

The final targets were purified by Prep-HPLC. The Prep-HPLC was carried out using the following method:

Method B: Prep-HPLC: Column, XBridge Shield RP18 OBD (19×250 mm, 10 um); mobile phase, Water (10 mmol/L NH4HCO3) and ACN, UV detection 254/210 nm.

NMR was recorded on BRUKER NMR 300.03 MHZ, DUL-C-H, ULTRASHIELD™300, AVANCE II 300 B-ACS™120 or BRUKER NMR 400.13 MHZ, BBFO, ULTRASHIELD™400, AVANCE III 400, B-ACS™120 or BRUKER AC 250 NMR instrument with TMS as reference measured in ppm (part per million).

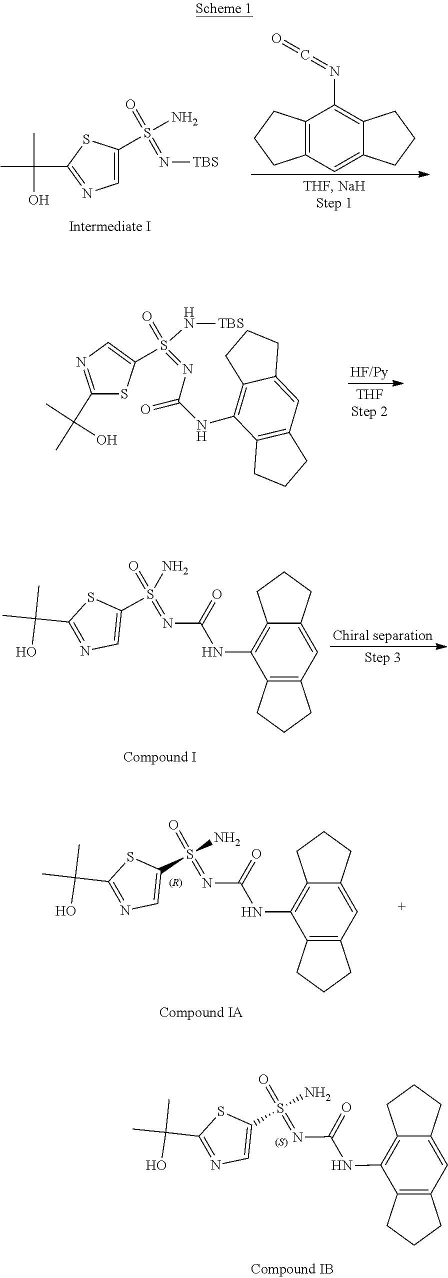

### Compound I

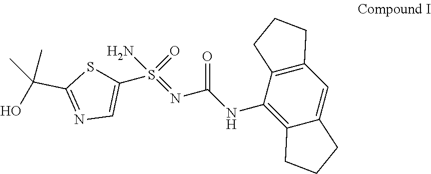 [SMILES: CC(C)(O)c1ncc(S(N)(=O)=NC(=O)Nc2c3c(cc4c2CCC4)CCC3)s1]

### Compound I: N′-(1,2,3,5,6,7-hexahydro-s-indacen-4-ylcarbamoyl)-2-(2-hydroxypropan-2-yl) thiazole-5-sulfonimidamide

### Step 1: N-(tert-butyldimethylsilyl)-N′-(1,2,3,5,6,7-hexahydro-s-indacen-4-ylcarbamoyl)-2-(2-hydroxypropan-2-yl) thiazole-5-sulfonimidamide

Into a 50-mL round-bottom flask was placed a solution of N′-(tert-butyldimethylsilyl)-2-(2-hydroxypropan-2-yl) thiazole-5-sulfonimidamide (Intermediate I) (336 mg, 1.0 mmol) in THF (10 mL). To this solution was added NaH (60% wt, 80 mg, 2.0 mmol) in portions at 0° C. The solution was stirred at 0° C. for 15 minutes, and this was followed by the addition of a solution of 4-isocyanato-1,2,3,5,6,7-hexahydro-s-indacene (209 mg, 1.1 mmol) in THF (5 mL) dropwise with stirring at RT. The resulting solution was stirred for 12 h at RT. The reaction was then quenched by the addition of 10 mL of NH4Cl (sat.). The resulting solution was extracted with 3×10 mL of DCM and the combined organic layers were concentrated under vacuum. This resulted in 535 mg (crude) of the title compound as a brown oil. MS-ESI: 535.0 (M+1).

### Step 2: N′-(1,2,3,5,6,7-hexahydro-s-indacen-4-ylcarbamoyl)-2-(2-hydroxypropan-2-yl) thiazole-5-sulfonimidamide

Into a 50-mL round-bottom flask was placed a solution of N-(tert-butyldimethylsilyl)-N′-(1,2,3,5,6,7-hexahydro-s-indacen-4-ylcarbamoyl)-2-(2-hydroxypropan-2-yl) thiazole-5-sulfonimidamide (535 mg, crude, 1.0 mmol) in THF (10 mL). To this solution was added HF/Py (70% wt, 143 mg, 5.0 mmol) dropwise at 0° C. The solution was stirred at RT for 4 h. The reaction was then quenched by the addition of 10 ml of water. The resulting solution was extracted with 3×10 mL of ethyl acetate and the combined organic layers were concentrated under vacuum. The crude product was purified by Prep-HPLC using Method B with ACN/water (20% to 60% in 10 minutes). This resulted in 189 mg (45%, 2 steps) of Compound I as a white solid.

Compound I: MS-ESI: 421.0 (M+1). 1H NMR (400 MHZ, DMSO-d6) δ 8.46 (br s, 1H), 8.04 (s, 1H), 7.80 (br s, 2H), 6.86 (s, 1H) 6.28 (s, 1H), 2.88-2.71 (m, 4H), 2.71-2.56 (m, 4H), 2.02-1.80 (m, 4H), 1.49 (s, 6H).

### Compound IA and Compound IB

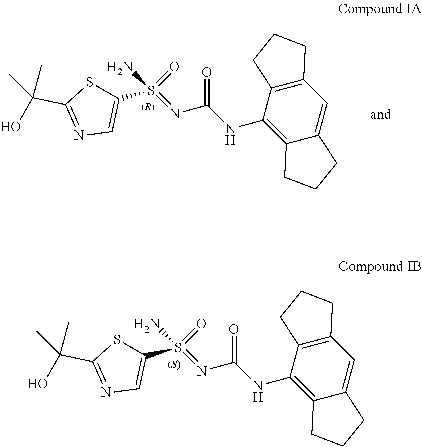

### Compounds IA and IB: (R) and(S)-N′-(1,2,3,5,6,7-hexahydro-s-indacen-4-ylcarbamoyl)-2-(2-hydroxypropan-2-yl) thiazole-5-sulfonimidamide

### Step 3: Chiral Separation

The Compound I product obtained as described in the previous step (189 mg) was resolved by Chiral-Prep-HPLC using the following conditions: Column, CHIRAL Cellulose-SB, 2*25 cm, 5 um; mobile phase, Hex (0.1% DEA) and EtOH (hold 20% EtOH over 16 min); Flow rate, 20 mL/min; Detector, UV 254/220 nm. This resulted in 70 mg of Compound IB (front peak, 99% ee) as a white solid and 65 mg of Compound IA (second peak, 97.5% ee) as a white solid.

Compound IB: MS-ESI: 421.0 (M+1). 1H NMR (400 MHZ, DMSO-d6) δ 8.43 (br s, 1H), 8.05 (s, 1H), 7.83 (br s, 2H), 6.87 (s, 1H) 6.29 (s, 1H), 2.82-2.71 (m, 4H), 2.71-2.56 (m, 4H), 2.02-1.80 (m, 4H), 1.50 (s, 6H).

Compound IA: MS-ESI: 421.0 (M+1). 1H NMR (400 MHZ, DMSO-d6) δ 8.41 (br s, 1H), 8.05 (s, 1H), 7.83 (s, 2H), 6.87 (s, 1H) 6.27 (s, 1H), 2.82-2.71 (m, 4H), 2.71-2.56 (m, 4H), 2.02-1.80 (m, 4H), 1.50 (s, 6H).

Intermediate I of scheme 1 was synthesized in accordance with the synthesis set out in WO2019/023147, and as provided in scheme 2, below.

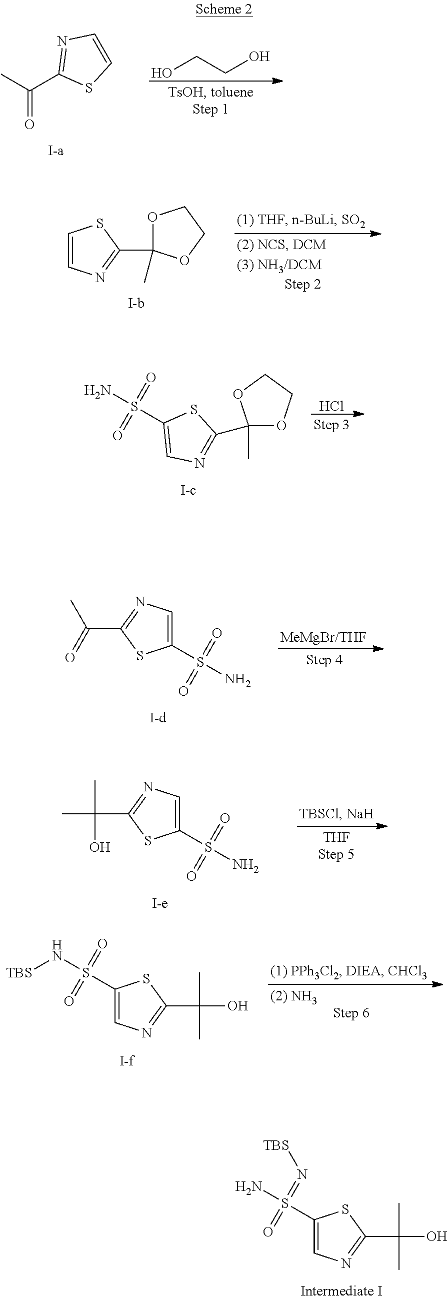

### Intermediate I

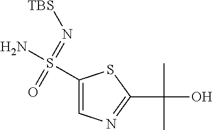

### N′-(tert-butyldimethylsilyl)-2-(2-hydroxypropan-2-yl) thiazole-5-sulfonimidamide

### Step 1:2-(2-Methyl-1,3-dioxolan-2-yl) thiazole

Into a 500-mL round-bottom flask was placed a solution of 1-(thiazol-2-yl) ethanone (20 g, 157.0 mmol) in toluene (300 mL) and ethane-1,2-diol (19.5 g, 314 mmol). To the solution was added TsOH (2.7 g, 15.7 mmol). The resulting solution was refluxed overnight and water was separated from the solution during the reflux. The resulting solution was diluted with 200 mL of water and extracted with 2×100 mL of ethyl acetate. The organic layers were combined, dried over anhydrous Na2SO4, and then concentrated under vacuum. This resulted in 26.6 g (99%) of the title compound as light yellow oil. MS-ESI: 172.0 (M+1).

### Step 2:2-(2-Methyl-1,3-dioxolan-2-yl) thiazole-5-sulfonamide

Into a 500-mL 3-necked round-bottom flask purged with and maintained under nitrogen was placed a solution of 2-(2-methyl-1,3-dioxolan-2-yl) thiazole (14 g, 81.6 mmol) in THF (200 mL). This was followed by the addition of n-BuLi (2.5 M in THF, 35.2 mL, 88.0 mmol) dropwise with stirring at −78° C. The resulting solution was stirred for 0.5 h at −78° C. and then SO₂ was introduced into the above reaction mixture. The reaction was slowly warmed to RT and then NCS (12.8 g, 95.86 mmol) was added. The resulting solution was stirred for 1 h at RT. The solids were filtered out. The resulting filtrate was concentrated under vacuum and then was diluted in DCM (160 mL). To the above was added a saturated solution of ammonia in DCM (300 mL). The resulting solution was stirred for 3 h at RT and then was concentrated under vacuum. The residue was applied onto a silica gel column and eluted with a gradient of ethyl acetate/petroleum ether (1:20 to 1:5). This resulted in 12.5 g (61%) of the title compound as a yellow solid. MS-ESI: 251.0 (M+1).

### Step 3:2-Acetylthiazole-5-sulfonamide

Into a 250-mL round-bottom flask was placed a solution of 2-(2-methyl-1,3-dioxolan-2-yl) thiazole-5-sulfonamide (12.5 g, 50.0 mmol) in THF (125 mL). To the above was added aq. HCl (4 N, 50.0 mL). The resulting solution was stirred for 6 h at 70° C. The resulting solution was diluted with 100 ml of water and extracted with 2×200 ml of ethyl acetate. The organic layers were combined, dried over anhydrous Na₂SO₄, then concentrated under vacuum. The residue was applied onto a silica gel column and eluted with a gradient of ethyl acetate/petroleum ether (1:2 to 1:1). This resulted in 9.3 g (90%) of the title compound as a yellow solid. MS-ESI: 207.0 (M+1).

Steps 4-6 used the same procedures as those specified for converting compound Z to compound Y shown in Scheme 3, to afford Intermediate I from compound I-d. MS-ESI: 336.1 (M+1).

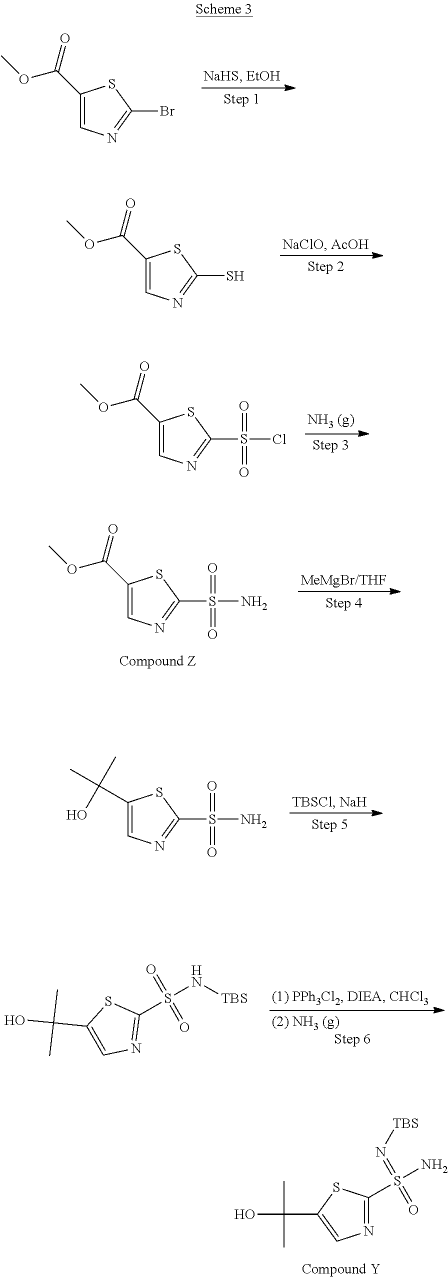

### Compound Y

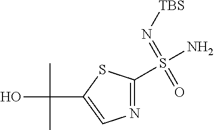

### N′-(tert-butyldimethylsilyl)-5-(2-hydroxypropan-2-yl) thiazole-2-sulfonimidamide

### Step 1: Methyl 2-mercaptothiazole-5-carboxylate

Into a 2-L round-bottom flask was placed methyl 2-bromothiazole-5-carboxylate (100 g, 450 mmol), EtOH (1000 mL), sodium hydrogensulfide (50 g, 890 mmol). The resulting solution was stirred for 2 h at 80° C. and then was cooled to 0° C. with a water/ice bath. The pH value of the solution was adjusted to 3 with hydrogen chloride (1 N). The solids were collected by filtration. This resulted in 63.2 g (80%) of the title compound as a light yellow solid. MS-ESI: 176.0 (M+1).

### Step 2: Methyl 2-(chlorosulfonyl) thiazole-5-carboxylate

Into a 1-L round-bottom flask was placed methyl 2-mercaptothiazole-5-carboxylate (30 g, 170 mmol) and acetic acid (300 mL). This was followed by the addition of sodium hypochlorite (300 mL, 8%-10% wt.) in portions at 0° C. The resulting solution was stirred for 2 h at RT and then was diluted with 500 mL of water. The solution was extracted with 3×300 mL of DCM and the combined organic layers were washed with 2×300 ml of brine and dried over anhydrous Na2SO4. The crude product as a yellow solution in DCM was used in the next step.

### Step 3: Methyl 2-sulfamoylthiazole-5-carboxylate

Into a 2-L round-bottom flask was placed methyl 2-(chlorosulfonyl) thiazole-5-carboxylate as a crude solution in DCM (900 mL). To the solution was introduced NH3 (g) below 0° C. for 20 minutes. The resulting solution was stirred for 1 h at RT and then concentrated under vacuum. The residue was applied onto a silica gel column and eluted with ethyl acetate/petroleum ether (1:5 to 1:3). This resulted in 23 g (75%, 2 steps) of the title compound as a white solid. MS-ESI: 223.0 (M+1).

### Step 4:5-(2-Hydroxypropan-2-yl) thiazole-2-sulfonamide

Into a 500-mL round-bottom flask purged with and maintained under nitrogen was placed a solution of methyl 2-sulfamoylthiazole-5-carboxylate (15 g, 67.5 mmol) in THF (150 mL). This was followed by the addition of MeMgBr/THF (3 M, 90 mL) dropwise with stirring at 0° C. The resulting solution was stirred for 14 h at RT and then was quenched by the addition of 100 mL of NH4Cl (sat.). The resulting solution was extracted with 3×150 ml of DCM. The organic layers were combined and dried over anhydrous Na2SO4, then concentrated under vacuum. The residue was applied onto a silica gel column and eluted with ethyl acetate/petroleum ether (1:5 to 1:3). This resulted in 11.5 g (78%) of the title compound as a white solid. MS-ESI: 223.0 (M+1), 221.0 (M−1) in positive and negative ion mode, respectively.

### Step 5: N-(tert-butyldimethylsilyl)-5-(2-hydroxypropan-2-yl) thiazole-2-sulfonamide

Into a 250-mL 3-necked round-bottom flask purged with and maintained under nitrogen was placed a solution of 5-(2-hydroxypropan-2-yl) thiazole-2-sulfonamide (5 g, 22.5 mmol) in THF (100 mL). Then to the above was added NaH (60% wt, 1.8 g, 45.0 mmol) in portions in an ice/water bath. After stirring for 20 minutes in a water/ice bath, this was followed by the addition of a solution of TBSCl (4.1 g, 27.2 mmol) in THF (10 mL) dropwise with stirring at 0° C. The resulting solution was stirred for 4 h at RT. The reaction was quenched with sat. NH₄Cl (100 mL). The resulting solution was extracted with 3×100 mL of ethyl acetate and the combined organic layers were dried over Na2SO4 and concentrated under vacuum. The crude solid was washed with ethyl acetate/hexane (1:5) (2×100 mL). This resulted in 6.81 g (90%) of the title compound as a yellow solid. MS-ESI: 337.1 (M+1), 335.1 (M−1) in positive and negative ion mode, respectively.

### Step 6: N′-(tert-butyldimethylsilyl)-5-(2-hydroxypropan-2-yl) thiazole-2-sulfonimidamide

Into a 100-mL 3-necked round-bottom flask purged with and maintained under nitrogen was placed a solution of PPh₃Cl₂ (3 g, 9.0 mmol) in CHCl₃ (100 mL). This was followed by the addition of DIEA (1.54 g, 11.9 mmol) dropwise with stirring at RT. The resulting solution was stirred for 10 min at RT. This was followed by the addition of a solution of N-(tert-butyldimethylsilyl)-5-(2-hydroxypropan-2-yl) thiazole-2-sulfonamide (2.0 g, 5.9 mmol) in CHCl₃ (30 mL) dropwise with stirring in an ice/water bath. The resulting solution was stirred for 30 min in an ice/water bath. To the above was introduced NH3 (g) below 0° C. for 15 minutes. The resulting solution was stirred for 20 minutes at RT. The solids were filtered out and the filtrate was concentrated and the residue was dissolved in 300 mL of ethyl acetate. The solution was washed with brine (2×100 mL), dried over Na₂SO₄ and concentrated under vacuum. The crude solid was washed with CHCl₃ (100 mL). Then the filtrate was concentrated under vacuum and the residue was further purified by a silica gel column with ethyl acetate/petroleum ether (1:10 to 1:3). The original washed solid and solid from silica gel purification were combined. This resulted in 1.2 g (60%) of the title compound as a white solid. MS-ESI: 336.1 (M+1). 1H-NMR (300 MHZ, DMSO-d6) δ 7.66 (s, 1H), 7.12 (s, 2H), 5.78 (s, 1H), 1.51 (s, 6H), 0.86 (s, 9H), 0.02 (s, 3H), 0.01 (s, 3H).

The following abbreviations have the indicated meanings:
ACN=acetonitrile
BTC=trichloromethyl chloroformate
Boc=t-butyloxy carbonyl
Davephos=2-Dicyclohexylphosphino-2′-(N,N-dimethylamino) biphenyl
DCM=dichloromethane
DEA=diethylamine
DMF=N, N-dimethylformamide
DMSO=dimethyl sulfoxide
DIEA=N, N-diisopropylethylamine
DPPA=diphenylphosphoryl azide
dppf=1,1′-Bis(diphenylphosphino) ferrocene
EtOH=ethanol
HATU=1-[Bis(dimethylamino)methylene]-1H-1,2,3-triazolo[4,5-b]pyridinium 3-oxid hexafluorophosphate
Hex=hexane
HPLC=high performance liquid chromatography
LC-MS=liquid chromatography-mass spectrometry
LiHMDS=lithium bis(trimethylsilyl)amide
LDA=lithium diisopropylamide
M=mol/L
Me=methyl
MeOH=methanol
MSA=methanesulfonic acid
NBS=N-bromosuccinimide
NCS=N-chlorosuccinimide
NMR=nuclear magnetic resonance
Pd (dppf) Cl₂=dichloro[1,1′-bis(diphenylphosphino) ferrocene]palladium
Ph=phenyl
PPh₃Cl₂=dichlorotriphenylphosphorane
Py=pyridine
RT=room temperature
Rt=Retention time
Rf=Retardation factor
Sat.=saturated
TBAF=tetrabutylammonium fluoride
TBS=tert-butyldimethylsilyl
TBSCl=tert-butyldimethylsilyl chloride
TBDPSCl=tert-butyldiphenylsilyl chloride
TEA=triethylamine
TFA=trifluoroacetic acid
THF=tetrahydrofuran
TLC=thin layer chromatography
TsOH=4-methylbenzenesulfonic acid
UV=ultraviolet
b.i.d.=twice daily
WCC=White Cell Count
EP=End Point
y=year
y/n=yes/no

### EXAMPLES

The following Examples illustrate the methods and uses described herein. They are not, however, intended to limit the scope of the described methods and uses in any way. Other variants of the embodiment will be readily apparent to one of ordinary skill in the art and are encompassed by the appended claims.

### Example 1: Clinical Study with Compound IA

The starting dose for Compound IA for participants enrolled in this trial, is set at 10 mg twice daily (b.i.d.) for two weeks to assess tolerability, followed by a single-step dose escalation to 25 mg b.i.d. for 10 weeks, for a total treatment period of 12 weeks. The initial dose level was selected primarily on data from the first-in-human (FIH) study (Example 2), in which skin rashes have been observed in some participants dosed once daily with 30, 100, and 200 mg Compound IA. The mechanism of the skin rash is yet unknown and b.i.d. dosing could potentially mitigate risk and is therefore being explored. In addition to assessing the tolerability of Compound IA, the two-week run-in period at a lower dose of 10 mg b.i.d. will also inform about the extent of peripheral PD marker inhibition.

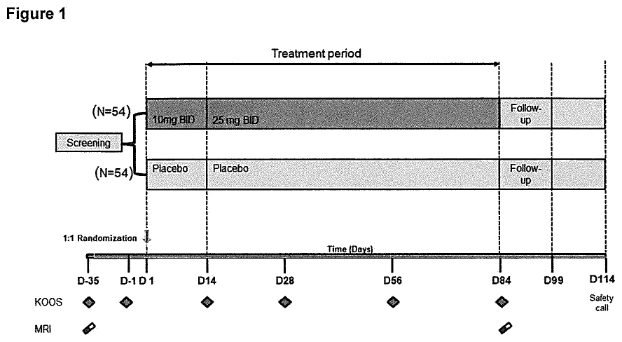

A randomized, two-arm, placebo-controlled, participant and investigator-blinded phase 2 study investigating the efficacy, safety and tolerability of Compound IA in patients with symptomatic knee osteoarthritis. FIG. 1 is a schematic overview of the treatment protocol with Compound IA (i.e. the R enantiomer of Compound I). In the first arm of the clinical study, 10 mg of Compound IA will be dosed orally twice daily for 13 consecutive days and 10 mg in the morning of Day 14, i.e. total dose of the first arm is 270 mg. The second arm of the study will begin directly after the first arm: 25 mg will be dosed in the evening of Day 14, and then 25 mg will be dosed twice daily for 69 days and in the morning of Day 84, i.e. the total dose of the second arm is 3500 mg.

In this Phase 2 study, the safety and tolerability of Compound IA in participants with symptomatic knee OA is to be evaluated and the efficacy of Compound IA in reducing knee pain is to be determined as evidenced by KOOS (knee injury and osteoarthritis outcome score).

The endpoint (EP) for the primary objective is the change from baseline in Knee injury and Osteoarthritis Outcome Score (KOOS) pain sub-scale at week 12.

The secondary objectives of the study are as follows:
To assess safety and tolerability of Compound IA;
To assess the efficacy of Compound IA on inflammatory joint structure features;
To assess the effect of Compound IA on systemic inflammatory status;
To assess pharmacokinetics of Compound IA in plasma;
To assess the efficacy of Compound IA in improving participants' report of knee symptoms and associated problems over time;
To assess the efficacy of Compound IA in improving participants' report of knee symptoms.

### The Endpoints (EP) for the Secondary Objectives are as Follows:

Safety endpoints (including vital signs, ECG parameters, safety laboratory assessment and adverse events);
Change from baseline in synovitis activity level measured from K^{trans} by DCE-MRI at week 12;
Change from baseline in serum high sensitivity C-reactive protein level and absolute neutrophil counts at week 2, 4, 8 and 12;
Change in plasma samples to quantify concentrations of Compound IA at various time points (week 2 and week 12) and to derive PK parameters in plasma (including but not limited to Cmax, AUC last, AUC0-12 h, and Ctrough);
Change from baseline in KOOS sub-scales (other symptoms, function in daily living, function in sport and recreation, knee-related quality of life) at weeks 2, 4, 8, and 12;
Change in numeric rating scale (NRS) for pain from baseline to weeks 2, 4, 8 and 12.

| Exploratory objectives | Endpoints for exploratory objectives |
|---|---|
| To explore the time course of | Biomarkers may include but are not |
| changes in exploratory biomarkers in | limited to NLRP3 pathway markers, e.g., |
| participants treated with COMPOUND IA | inflammatory chemokines/cytokines (e.g., |
| vs. placebo | CXCL10, IL1b, IL6, TNF-α and IL18) in serum |
| | and synovial fluid and Caspase 1 in serum. |
| To explore the molecular profile | Potential protein signatures in |
| and pathways involved in the response to | synovial fluid and serum, assessed by |
| COMPOUND IA, and to correlate markers | SomaScan protein profiling at week 12 |
| with other clinical readouts (i.e. synovitis; | (synovial fluid) or baseline and week 12 |
| pain) | (serum) |
| To assess exposure of | Ctrough of COMPOUND IA in |
| COMPOUND IA in synovial fluid | synovial fluid and synovial fluid-to-plasma |
| | concentration ratio at week 12 |
| To explore PK/PD relationship of | Plasma concentrations of |
| COMPOUND IA in plasma in participants | COMPOUND IA and exploratory biomarkers |
| with OA | (e.g. IL1b, IL6, IL18, or CXCL10) or |
| | inflammatory markers such as hsCRP in |
| | circulation |
| To explore the efficacy of | Change from baseline in cartilage |
| COMPOUND IA vs. placebo on changes | volume and thickness of the index region |
| in cartilage structure and bone marrow | measured by MRI at week 12. |
| lesions | |
| To assess the efficacy of | Change from baseline in effusion |
| COMPOUND IA vs. placebo on additional | volume measured by MRI at week 12 |
| inflammatory features | |
| To assess the effect of | Levels of exploratory markers in urine |
| COMPOUND IA vs. placebo on renal | (such as, but not limited to, KIM-1, NGAL) to |
| injury markers | characterize the type and extent of kidney |
| | impairment, gated upon evidence of signature |
| | of potential renal impairment based on |
| | established clinical assessments. |
| To explore whether individual | Differences between different |
| variation in genes related to drug | CYP2C9 metabolizers in PK parameters of |
| metabolism (e.g., CYP2C9 genotypes), | COMPOUND IA such as AUClast and Cmax |
| the drug target pathway, or other relevant | (if feasible) |
| genetic pathways confer differential | Due to the exploratory nature of this |
| exposure or response to the COMPOUND | objective, other specific endpoints are not |
| IA | predefined. |
| To explore the effect of | Quantifiable variables of physical |
| COMPOUND IA vs. placebo on changes | activity corrected for wearing time, using a |
| in physical functioning | mobility tracker device |
| To assess biomarkers for | Normalization biomarkers may include |
| normalization of synovial fluid to systemic | but are not limited to total protein (synovial |
| levels | fluid collection at week 12) and urea. |
| To explore minimally invasive | Levels of select biomarkers (e.g., |
| biomarker sampling methods (e.g., | hsCRP) in transdermal blood samples. |
| transdermal blood samples) for select | |
| biomarkers | |
| To assess the impact of participant | Placebell © ™ composite covariate |
| personality on response to treatment. | (placebo score) calculated for each |
| | participant by integrating participant |
| | personality characteristics (captured with |
| | MPsQ questionnaires) and other pertinent |
| | baseline features. |

### Study Design:

This study uses a randomized, 2 treatment arm, parallel-group, participant and investigator-blinded, placebo-controlled design to evaluate the safety and tolerability of oral Compound IA in approximately 108 participants with symptomatic, inflammatory knee OA, and determining efficacy of Compound IA as evidenced by reduction in knee pain by KOOS (knee injury and osteoarthritis outcome score) after 12 weeks of treatment.

The study consists of a screening period of up to 45 days, used to assess eligibility and to taper participants off disallowed medications. At Day 1 visit, eligible participants will be randomized to one of the treatment arms. Eligible participants will enter the treatment period, which will begin with a 2-week titration period where they will receive Compound IA 10 mg twice daily or placebo orally for 14 consecutive days, followed by a 10-week treatment period where they will receive Compound IA 25 mg twice daily or placebo orally. An end of study visit will occur 15 days after the last dose and a post study safety contact will occur 30 days after last dose. The total study duration from screening until end of study is expected to be a maximum of 19 weeks.

The assessment to address the primary objective will be performed at the end of the treatment period (week 12).

### The Study Involves Three Periods:

Screening period: The screening period consists of 2 visits, a screening visit and a baseline visit.
Treatment period: The treatment period will consist of 5 visits.
Follow up period: The patients will be followed-up at the end of the study visit, approximately 15 days after last dose. Furthermore, a safety follow-up call will be performed approximately 30 days after last dose.

### Treatment Period:

### The Treatment Period Will Consist of 5 Visits:

Treatment initiation visit (Day 1): Participants who meet all inclusion and no exclusion criteria, will be enrolled and will begin taking a total daily dose of 20 mg (10 mg b.i.d.) of COMPOUND IA or matching placebo tablets twice daily for 14 consecutive days (last dose on Day 14, morning dose). The first dose, either COMPOUND IA 10 mg or placebo, will be administered and study treatment will be dispensed to the participant for continued treatment at home. Participants may be domiciled the evening prior to a scheduled visit for their convenience and logistical aspects, at the discretion of the participant and investigator.
Participants will be evaluated on Day 14 as outlined in the assessment schedule. Provided the treatment was well tolerated based on the Investigator's judgement and guidance, they will start taking a total daily dose of 50 mg (25 mg b.i.d.) of COMPOUND IA or matching placebo tablets b.i.d. starting on Day 14 (evening dose only) for 10 weeks. The last dose will be administered on Day 84 (morning dose only).
Participant will have assessments on Day 28, Day 56, and Day 84. The site staff will perform phone calls at least once between two monthly visits to remind participants to take their study treatment.

### Follow Up Period:

Participants will be followed-up for an end of study evaluation at the End of study visit, approximately 15 days after last dose (Day 99). A safety follow-up call will be performed approximately 30 days after the last dosing (Day 114) to record any potential safety event.

### Inclusion Criteria:

Participants eligible for inclusion in this study must meet all of the following criteria:
1. Written informed consent must be obtained before any assessment is performed.
2. Able to communicate well with the investigator, to understand and comply with the requirements of the study.
3. Male and female participants>=50 and <=80 years old on the day of Informed Consent signature.
4. Participants must weigh at least 50 kg to participate in the study, and must have a body mass index (BMI) within the range of 18-35 kg/m2 at screening. BMI=Body weight (kg)/[Height (m)]2
5. High sensitivity C-reactive protein (hsCRP)>=1.8 mg/L at screening
6. Symptomatic OA with pain (Numeric Rating Scale [NRS] 5-9, inclusive) in the target knee for the majority of days in the last 3 months prior to screening. At Screening, the patient will be given a diary to record pain and use of analgesic medications. The participant must be compliant with filling in the diary at least 5 out of 7 days prior to Baseline, and have PRO reported NRS Pain≥5 to ≤9 at Screening and Baseline.
7. Primary source of pain is due to OA in target knee based on widespread pain index (WPI) score<=4 at screening
8. KOOS pain sub-scale score<=60 in index knee at screening and baseline.
9. Radiographic disease: K&L grade 2 or 3 knee osteoarthritis in the target knee, as per the OARSI Atlas, confirmed by X-ray at screening.
10. Active synovial inflammation at screening, defined as either moderate (score 9-12) or severe (score>=13) based on contrast enhanced MRI (CE-MRI) of the whole knee for synovitis detection from 11 sites Guermazi et al 2011.
11. Diagnosis of primary tibiofemoral knee OA by standard American College of Rheumatology (ACR) clinical and radiographic criteria at Screening
12. Current use of analgesic therapy for control of local pain in the target knee:
   Patients taking paracetamol/acetaminophen, including combination drugs containing low dose opioids, can continue using this as per the package insert/doctor's instruction
   Patients taking any other analgesic medication, including NSAIDs and selective COX-2 inhibitors but excluding topical NSAIDs or steroids, for any pain indication including knee pain, must be willing to switch at Screening to paracetamol/acetaminophen, including combination drugs containing low dose opioids, as per the package insert/doctor's instruction. NSAIDs are allowed only as rescue medication, but must not be used within 48 hours or five half-lives, whichever is longer, prior to any PRO assessment.
   Patients taking glucosamine or chondroitin must be willing to discontinue these from Screening.

### Key Exclusion Criteria:

Participants meeting any of the following criteria are not eligible for inclusion in this study.
1. Total WBC count<3,000/μL, absolute peripheral blood neutrophil count (ANC)<1,000/μL, hemoglobin<8.5 g/dL (85 g/L) or platelet count<100,000/μL at Screening
2. Known autoimmune disease with inflammatory arthritides (including but not limited to rheumatoid arthritis, psoriatic arthritis, ankylosing spondylitis, systemic lupus erythematosus), crystal-induced arthritides (gout, pseudogout associated arthritis), active acute or chronic infection or past infection of the knee joint, Lyme disease involving the knee, reactive arthritis, systemic cartilage disorders, moderate to severe fibromyalgia (widespread pain index, WPI, >4 out of 19), or a known systemic connective tissue disease.
4. Metabolic or genetically-based abnormalities associated with arthropathy.
5. Participant has an unstable target knee joint, knee hardware or insufficiently reconstructed ligaments based on medical history and physical examination by the Investigator at screening.
6. Participant has symptomatic, isolated patello-femoral pain in the index knee as per the Investigator's examination at screening.
7. Use of electrotherapy, acupuncture, and/or chiropractic treatments for knee OA within 4 weeks prior to screening.
8. Any known active infections, including skin or knee infections or infections that may compromise the immune system, such as HIV or chronic hepatitis B or C infection. COVID-19 specific: It is highly recommended that PCR or antigen testing for COVID-19 be completed within 1 week prior to first dosing. If testing is performed, negative test results are required prior to enrolment into the study. Additional testing may occur at the discretion of the investigating physician. COVID-19 testing should be completed via nasal or throat swabs. If testing is not performed, the investigator must document their discussion with the participant regarding testing, and the rationale for not testing, in the source documentation. This requirement may be ignored if the pandemic is declared ended by the country where the site is located, and resumed if the pandemic recurs.
9. Any diagnosed psychiatric condition that includes, but is not limited to, a history of mania, bipolar disorder, psychotic disorder, schizophrenia, or schizoaffective disorder, depression or anxiety which may jeopardize participant safety or compliance with study procedures, as judged by the investigator.
10. History of lymphoproliferative disease or any known malignancy or history of malignancy of any organ system within 5 years of screening (except for basal cell carcinoma or actinic keratoses that have been treated with no evidence of recurrence in the 3 months prior to screening, or carcinoma in situ of the cervix or non-invasive malignant colon polyps that have been removed).
11. Symptomatic hip OA or hip prosthesis recently implanted (within 1 year prior to screening) or foreseen within the study period (either side).
12. Other pathologies affecting the knee, including subchondral insufficiency fractures, bone fracture (acute or subacute in less than 6 months prior to screening) or bone bruise, osteonecrosis, osteochondral lesion, malignant bone marrow infiltration, solid tumors, meniscus extrusion greater than 50% and/or macerated meniscus and/or patellofemoral dysplasia based on clinical or imaging assessments.
13. Unstable target knee joint (including, but not limited to, posttraumatic or congenital laxity) or insufficiently reconstructed ligaments based on medical history and/or physical examination by the Investigator.
14. Use of prohibited medications: any local i.a. treatment into the knee, including but not restricted to viscosupplementation and corticosteroids within 12 weeks prior to Day 1; long-term treatment (>14 days) with oral corticosteroids>5 mg/day within 4 weeks prior to Day 1; oral glucosamine, chondroitin sulfate, or any nutraceutical with potential activity on cartilage repair within 2 weeks prior to Day 1; systemic Non-Steroidal Anti-Inflammatory Drugs (NSAIDs) or selective COX-2 inhibitors within 48 hours or five half-lives, whichever is longer, from PRO assessments; any other immunomodulatory drugs or treatment which cannot be discontinued or switched to a different medication within 28 days or 5 half-lives of screening (whichever is longer if required by local regulations), or until the expected PD effect has returned to baseline.
15. Severe malalignment greater than 7.5 degrees in the target knee (either varus or valgus), measured using X-ray at screening.
16. Participant unable or unwilling to undergo MRI or having contraindications to MRI (e.g., metallic implants, metallic foreign bodies, pacemaker, defibrillator) or to the use of gadolinium-based agents, e.g., patients with previous severe allergic/anaphylactoid reaction to a gadolinium-based contrast agent; patients with severe renal disease (eGFR<60 mL/min calculated using the CKD-EPI formula [https://www.kidney.org/professionals/KDOQI/gfr_calculator] or >=2+ protein on urine dipstick testing at screening and at baseline), or acutely deteriorating renal function.
17. Moderate to severe pain in the contralateral knee for the majority of days in the last 3 months prior to screening, as per patient judgement.
18. History of, or planned, knee replacement (partial or total) in either knee. Any other previous surgical intervention in the target knee including mosaicplasty, microfracture, meniscectomy>50% or osteotomy. Arthroscopy or lavage of the target knee within six months prior to screening, or planned during the study.
19. Women of child-bearing potential, defined as all women physiologically capable of becoming pregnant, unless they are using highly effective methods of contraception during dosing and for 15 days after stopping of investigational drug.
20. Pregnant or nursing (lactating) women.
21. History or current diagnosis of ECG abnormalities indicating significant safety risk for participants participating in the study such as:
Concomitant clinically significant cardiac arrhythmias, e.g. sustained ventricular tachycardia, and clinically significant second or third degree AV block without a pacemaker.
History of familial long QT syndrome or known family history of Torsades de Pointes.
22. History of drug abuse or unhealthy alcohol use within the 12 months prior to expected first dose, or evidence of such abuse as indicated by the laboratory assays conducted at screening visit.
23. History of hypersensitivity to any of the study treatments or excipients or to drugs of similar chemical classes.
24. Use of other investigational drugs within 5 half-lives of enrollment, or until the expected pharmacodynamic effect has returned to baseline, whichever is longer.
25. If, in addition to primary knee osteoarthritis, spine/hand/shoulder/hip/foot/other primary osteoarthritis is present, it should have been present for at least 3 months prior to screening and should be documented with a diagnosis and symptoms as by investigator's judgements.
26. Secondary osteoarthritis with history or any evidence in the potential target joint of the following diseases: septic arthritis, inflammatory joint disease, gout, recurrent episodes of pseudogout, Paget's disease of bone, articular fracture, ochronosis, acromegaly, hemochromatosis, Wilson's disease, primary osteochondromatosis, heritable disorders, collagen gene mutations.
27. Participants receiving concomitant medications that are known to be strong or moderate inducers of cytochrome CYP2C9 enzyme and/or strong inhibitors of CYP2C9 and/or strong inducers of CYP3A and the treatment cannot be discontinued or switched to a different medication within 5 half-lives or 1 week (whichever is longer) prior to Day1 and for duration of the study.
28. History of clinically significant liver disease or liver injury as indicated by abnormal liver function tests (as defined below) including but not limited to SGOT (AST), SGPT (ALT), alkaline phosphatase, serum bilirubin, albumin and prothrombin time. The Investigator should be guided by the following criteria:
   Any single parameter may not exceed 2× upper limit of normal (ULN).
29. Participants with the CYP2C9*3/*3 genotype defined as homozygous carriers of the CYP2C9*3 allele.
30. Onset of symptoms, or diagnosis, of primary osteoarthritis in other than the knee joints<3 months prior to screening.
31. Live vaccines within 4 weeks of Day 1 (i.e. first dose of COMPOUND IA).
32. Known history of renal disease including nephrolithiasis.

### Efficacy Assessments:

The efficacy assessments described in this section will be evaluated in all participants in both treatment arms. Pain (primary endpoint) will be assessed by Patient Reported Outcomes (PROs). Pharmacodynamic samples will be collected.

Synovitis (secondary endpoint), articular cartilage volume/thickness and effusion volume (exploratory endpoints) will be evaluated from MRI.

Pharmacodynamic (PD) samples will be obtained and evaluated in all participants at all dose levels, including the placebo group.

### Patient Reported Outcomes (PROs):

The participant will be given the PRO measure(s) to be completed at the scheduled visit before other clinical assessments are conducted. The questionnaires should be completed in the language most familiar to the participant. The participant should be given sufficient space and time to complete the PRO measure(s). A participant's refusal to complete all or any part of a PRO measure should be documented in the Case Report/Record Form (CRF). Study staff should check the PRO measure(s) collected for completeness and ask the participant to complete any missing responses. Completed PROs, including any unsolicited comments written by the participant, must be reviewed and assessed by the investigator for responses which may include potential AEs or SAEs before any clinical study examinations are conducted. If AEs or SAEs are confirmed, then study investigators should not encourage the participant to change responses reported in the completed questionnaires.

### Knee Injury and Osteoarthritis Outcomes Score (KOOS):

Knee-related pain will be assessed as the primary endpoint by means of the Knee Injury and Osteoarthritis Outcomes Score (KOOS) measure collected at regular intervals (Roos E M, Davis A M (2012) Recommendations for publication of cross-cultural validation studies of patient-reported outcomes (PROs) in Osteoarthritis and Cartilage. Osteoarthritis Cartilage. p. 4-5.). The KOOS includes 42 items grouped into five sub-scales: Pain, Other Symptoms, Activities of Daily Living (ADL), Function in Sport and Recreation (Sport/Rec), and Knee-related Quality of Life (QoL). Each sub-scale is scored separately on a scale from 0 to 100, with a higher number indicating better condition (Collins N J, Misra D, Felson D T, et al (2011) Measures of knee function: International Knee Documentation Committee (IKDC) Subjective Knee Evaluation Form, Knee Injury and Osteoarthritis Outcome Score (KOOS), Knee Injury and Osteoarthritis Outcome Score Physical Function Short Form (KOOS-PS), Knee Outcome Survey Activities of Daily Living Scale (KOS-ADL), Lysholm Knee Scoring Scale, Oxford Knee Score (OKS), Western Ontario and McMaster Universities Osteoarthritis Index (WOMAC), Activity Rating Scale (ARS), and Tegner Activity Score (TAS). Arthritis Care Res (Hoboken). p. S208-28). The PRO KOOS score is an expanded version of the WOMAC score, which traditionally has been used in OA clinical trials (KOOS User Guide 2003). KOOS includes WOMAC OA Index LK3.0 in its complete and original format and WOMAC scores are able to be calculated. Therefore, the KOOS score provides a more comprehensive assessment as it also includes functioning in sport and recreation, as well as knee-related quality of life. KOOS requires approximately 10 minutes for participants to complete.

### Numerical Rating Scale (NRS):

The Numerical Rating Scale (NRS) for Pain (Hawker G A, Mian S, Kendzerska T, et al (2011) Measures of adult pain: Visual Analog Scale for Pain (VAS Pain), Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), and Measure of Intermittent and Constant Osteoarthritis Pain (ICOAP). Arthritis Care Res (Hoboken). p. S240-52) with a recall period of 24 hours and traditionally used to assess pain in clinical trials, is assessed at regular intervals to confirm eligibility and to evaluate pain status throughout the study.

### Widespread Pain Index (WPI):

Widespread Pain Index (WPI) is assessed at screening only to exclude participants with substantial pain originating from other regions than the target knee, fibromyalgia, or other undiagnosed diseases which may interfere with pain assessment.

### Pain Diary:

A pain diary will also be completed by the participant daily during the 12-weeks study and transferred to the CRF at each visit, beginning with Screening. This diary will be used to record basic pain medication, rescue medication and pain levels, daily. The participant may choose when, but should assess their pain intensity at approximately the same time every day. The NRS pain assessment in the diary and the NRS pain assessment performed during study visits should be documented separately in the CRF. At each study visit from Screening to Day 84, participants must be provided with a new pain diary that covers at least the period until the next planned visit. Prescription or use of pain medications will still need to be documented as Concomitant Medications according to Section 6.2.1.

### Knee MRI:

MRI will be obtained from the target knee to select participants with active synovitis and visualize the cartilage and other structures through the knee. The imaging protocol will be developed to quantify changes in synovitis, effusion volume, and volume and thickness of cartilage in the index region (i.e., the region where most cartilage damage occurs in OA participants with KL 2-3) during treatment. The index region is defined as the union of the femoral medial anterior (FMA), central (FMC) and posterior (FMP) cartilage sub-regions in the knee. This approach will demonstrate the effectiveness of COMPOUND IA in reducing knee inflammation and whether this response is correlated with a decrease in pain. It will also be used to quantify changes in volume and thickness of cartilage in the index region. In addition, the assessment of the synovitis activity level using a dynamic-contrast-enhanced (DCE) MRI approach will demonstrate the effectiveness of COMPOUND IA in reducing knee inflammation and whether this response is correlated with a decrease in pain.

The safety of the treatment period is supported by 13-week GLP toxicology studies in rat and cynomolgus monkey. The expected mean steady-state daily systemic drug exposure at 25 mg b.i.d. dosed with food in participants will remain about 7-fold lower than the mean NOAEL plasma AUC in rat with even larger safety margins on free AUC (14-fold) or Cmax (17-fold (total) and 34-fold (free)). Moreover, the expected mean steady-state drug exposure (total and unbound) will remain at least 49-fold lower than the exposure noted at NOAEL dose of 150 mg/kg/day (highest tested dose) in monkeys.

Safety margins for COMPOUND IA dosed at 25 mg b.i.d. based on 13-week GLP toxicology studies:

| | | | | Exposure | | Exposure | |
|---|---|---|---|---|---|---|---|
| | | | Total or | multiples | | multiples | |
| Human | Predicted human | | unbound | RAT NOAEL | | MONKEY NOAEL | |
| Dose | PK, steady-state | | exposure | based on: | | based on: | |
| | Cmax,ss | AUC0-24 h, | | Cmax | AUC0-24 h | Cmax | AUC0-24 h |
| | (μg/mL) | ss (μg * h/mL) | | | | | |
| 25 mg | 3.15 | 58 | Total | 17 | 7 | 82 | 61 |
| b.i.d./ | | | Unbound^{a} | 34 | 14 | 66 | 49 |
| crystalline | | | | | | | |
| tablet with | | | | | | | |
| food | | | | | | | |

^{a}corrected for plasma protein binding

### Drug-Drug Interaction Issues:

Evaluation and recommendations for drug-drug interaction clinical studies of cytochrome P450 (CYP) substrates/modulators and Compound IA are based on in vitro/preclinical data and physiology-based PK simulations. Compound IA is expected to be eliminated mainly via hepatic CYP-mediated metabolism with CYP2C9 (68%) and CYP3A4 (29%) as the main contributing enzymes. Participants who are poor CYP2C9 metabolizers will be excluded from this study.

Considering treatment duration and sufficient safety margins, administration of Compound IA is considered safe even under conditions of elevated exposures to Compound IA.

### Prohibited Drugs and Herbal Medications:

Anti-rejection/immune modulatory therapies (e.g., anakinra, canakinumab or other investigational IL-1/NLRP3 binding or blocking therapy)
Live vaccine
Strong or moderate inducers of CYP2C9 or strong inducers of CYP3A including carbamazepine, enzalutamide, lumacaftor, phenobarbital, phenytoin, rifabutin, mitotane and St. John's wort (Hypericum perforatum)
Strong inhibitors of CYP2C9 including miconazole, berberine (herbal product), sulfaphenazole, fluconazole, resveratrol (herbal product)
Other investigational products
   Drugs to be Used with Caution:
Drugs that are metabolized by CYP3A: In vitro metabolism studies showed that Compound IA might have the potential to induce the metabolism of drug substrates metabolized by isoenzyme CYP3A. Therefore, investigators, at their discretion, may administer concomitant medications known to be metabolized by CYP3A4/5. Patients receiving such medications may require dose titration or increase of the concomitant drug. Particularly, caution is advised when Compound IA is co-administered with drugs that are sensitive substrates of CYP3A and/or have a narrow therapeutic index.
Drugs that are strong or moderate inhibitors of CYP3A: Compound IA was identified in vitro as a substrate of CYP3A, so an increase in systemic exposure of Compound IA when co-administered with strong CYP3A inhibitors such as antiviral drugs (e.g., ritonavir), antifungal (e.g., itraconazole, ketoconazole) and antibiotics (e.g., erythromycin, clarithromycin) cannot be ruled out. Investigators may, at their discretion, co-administer known inhibitors of CYP3A, but their duration should be kept as short as possible, and patients must be closely monitored.

### Example 2: Clinical First-In-Human (FIH) Study

### Study Design

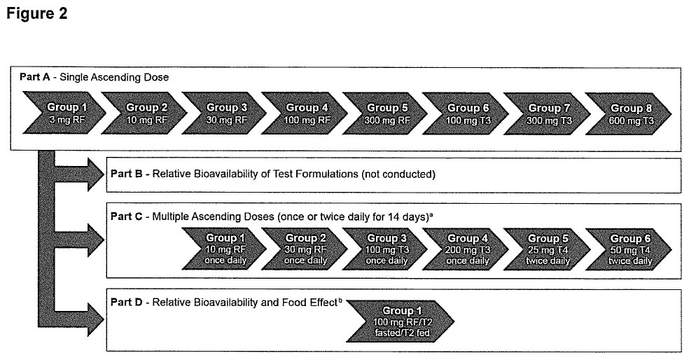

The study design comprised of 4 parts: single ascending dose (SAD; Part A), relative bioavailability of tablet formulations (Part B), multiple ascending dose (MAD; Part C), and relative bioavailability and food effect (Part D) (FIG. 2; RF=Reference Formulation (crystalline suspension); T2=Test Formulation 2 (crystalline tablet); T3=Test Formulation 3 (spray-dried dispersion suspension); T4=Test Formulation 4 (encapsulated crystalline tablet)). In each group of Parts A and C, 8 subjects were randomized in a 3:1 ratio to receive Compound IA (6 subjects) or matching placebo (2 subjects).

For Part A, eight cohorts of eight eligible subjects were enrolled. Each subject received a single oral dose of Compound IA (3, 10, 30, 100, 300 mg of crystalline suspension and 100, 300, 600 mg of spray-dried dispersion (SDD) under fasted conditions. As this was a FIH study, two sentinel subjects were dosed first, at least 24 hours before the rest of the cohort was dosed, to assure maximum safety. Part B was skipped because the data from Part A provided for an adequate comparison of crystalline and SDD formulations.

For part C, eligible subjects were enrolled in six different cohorts. Each subject received once daily (QD) multiple doses of Compound IA (10, 30 mg of crystalline suspension and 100, 200 mg of SDD for 14 days) in fasted condition, and (25, 50 mg of encapsulated crystalline tablet for 13 days and single dose on Day 14 or placebo) under fed condition. Subjects were dosed in Part C following review available safety, tolerability and PK data from preceding groups in Part A.

Part D had an open-label, randomized, 3-period crossover design consisting of 1 group of 6 subjects. The PK of the crystalline tablet formulation of Compound IA was compared between fed and fasted conditions, and with the PK of the crystalline suspension of Compound IA under fasted conditions. Subjects received 3 doses of Compound IA with washout period of 7-14 days between each dose (Dose 1:100 mg oral suspension in fasted condition; Dose 2:100 mg oral tablet in fasted condition; Dose 3:100 mg oral tablet in fasted condition). Based on these doses, subjects were randomly assigned to 1 of 6 treatment sequences (1 subject per sequence) prepared using Williams design.

### Subjects

Eligible subjects were healthy male, and female aged between 18 to 64 years with body mass index (BMI)≥18.5 and ≤30.0 kg/m². No subject participated in more than 1 part or group. A written informed consent was obtained prior to any study procedure. Subjects participating in Part D had to be willing and able to consume the entire high-fat breakfast meal in the designated timeframe. Subjects were excluded if they had history of major psychiatric disorders, diagnosis of intellectual disability, clinically significant vital signs abnormality, and using tobacco products within 90 days prior to (the first) drug administration through follow-up.

### Blinding

In Part A and C, active and placebo treatments could not be distinguished based on labelling, were identical in appearance, and were similar in taste and smell. To maintain the blind, the same number of tablet or suspension was given to each subject in respective cohort. The investigator and subjects remained blinded throughout the relevant part of the study, and the blind remained unbroken throughout. The Sponsor (IFM Management, Inc.) became unblinded with access to all study data and was provided with a copy of the randomization codes to support decision making concerning the study. The Part D was open label, only Compound IA was administered in subjects to 1 of 6 treatment sequences (1 subject per sequence) according to a Williams design.

### Objectives

The primary objective of the study was to evaluate the safety and tolerability of SAD and MAD oral doses of Compound IA in healthy subjects in all parts of the study. Key secondary objectives were to characterize the PK profile following single and multiple doses of Compound IA and to evaluate the effect of food on PK profile of Compound IA.

### Assessments

Safety assessments in all parts of the study included adverse event (AE) reporting using the Medical Dictionary for Regulatory Activities (version 22.1), clinical laboratory tests (biochemistry, hematology, and urinalysis), vital signs, electrocardiograms (ECGs), physical examination and skin biopsy (as applicable).

In single-dose part, blood samples were collected for determining the concentrations of Compound IA at the following time points relative to dosing on Day 1: at pre-dose and 0.25, 0.5, 0.75, 1, 1.5, 2, 3, 4, 6, 8, 12, 24, 36, and 48 hours post-dose, and at the follow-up visit. In multiple-dose part, relative to dosing on Days 1 and 14, samples were collected at pre-dose and 0.25, 0.5, 0.75, 1, 1.5, 2, 3, 4, 6, 8, and 12 hours post-dose; on Days 2, 4, 7, 9, and 11: at pre-dose; after the last dose on Day 14: at 24 and 36 hours (Day 15), and 48 hours (Day 16) post-dose; and at the follow-up visit. Following PK parameters were estimated using noncompartmental analysis maximum concentration in plasma (Cₘₐₓ); time to maximum concentration (tₘₐₓ); concentration at 24 h post-dose (C₂₄ₕ) (Part A only); Lag time: time of observation prior to the first quantifiable concentration (t_{tag}); time of the last quantifiable concentration (tₗₐₛₜ); area under the concentration-time curve from time 0 to the last quantifiable concentration (AUC₀₋ₗₐₛₜ); area under the plasma concentration-time curve from time 0 to infinity (AUC_{0-inf}); area under the plasma concentration-time curve from time 0 to 24 hour post-dose (AUC₀₋₂₄); terminal phase rate constant (Kₑₗ); terminal phase half-life (t_{1/2}); apparent total body clearance (CL/F); and apparent volume of distribution at terminal phase (V_{z}/F); and in addition for Part C only: area under the plasma concentration-time curve over the dosing interval from time 0 to 12 hours post-dose (AUC₀₋ₜₐᵤ); apparent clearance at steady state (CLss/F); accumulation ratio based on AUC₀₋ₜₐᵤ (R_{ac}, AUC); and accumulation ratio based on Cₘₐₓ (R_{ac}, Cₘₐₓ).

To determine the PD response to NLRP3 inhibition, whole blood samples were collected for exploratory PD analysis (Part A and Groups 1-3 of Part C). An ex-vivo stimulation by activating the NLRP3 inflammasome with lipopolysaccharide (LPS) was evaluated compared to control conditions, followed by analysis of blood cell release of the inflammatory marker IL-1β.

### Statistical Analysis

All data were summarized using descriptive statistics and were listed and summarized in tabular and/or graphical form. Descriptive statistics for all relevant PK parameters included: n, arithmetic mean, standard deviation (SD), coefficient of variation (CV %), minimum, median, maximum, geometric mean, and geometric CV %. For tₘₐₓ only median, minimum, and maximum are presented. PK parameters were calculated using noncompartmental methods using software Phoenix Version 8.1. Concentrations below the lower limit of quantification (LLOQ) were treated as zero in summary statistics for concentration data only. The linear trapezoidal rule was used for AUC calculation. Regression analysis of the terminal plasma elimination phase for the determination of t_{1/2} included at least 3 data points after Cₘₐₓ. Parameters with an adjusted r² below 0.80 were flagged but included in the descriptive statistics. The parameters AUC_{0-inf}, % AUCₑₓₜᵣₐ, CL/F, and VZ/F with an % AUCₑₓₜᵣₐ above 20% were excluded from the descriptive statistics.

In Part A, dose proportionality was explored using a regression power model relating logarithmically (log)-transformed Cₘₐₓ, AUC₀₋ₗₐₛₜ, and AUC_{0-inf} to the log-transformed dose level. Point estimates for the intercept and the slope and corresponding 90% confidence intervals (CIs) for the slope were calculated. For Part C, dose proportionality was not explored. In Part D, the relative bioavailability of the Test Formulation (crystalline tablet, SDD and crystalline tablet) versus the Reference Formulation (crystalline suspension), as well as the effect of food, was explored using an analysis of variance (ANOVA) model on the PK data.

For the following treatments, the least-squares geometric mean ratios were presented together with 90% CIs: 100 mg Compound IA tablet fasted over 100 mg Compound IA suspension fasted, and 100 mg Compound IA tablet fed over 100 mg Compound IA tablet fasted.

Combined individual and mean plots of the individual IL-1β concentrations versus time are presented by treatment. Modeling of Compound IA effects on a corrected, stimulated ex vivo Lipopolysaccharide (LPS) challenge in whole blood included evaluation of the relationship between LPS challenge results by conditionally weighted residual modeling.

### Results

### Subject Disposition and Demographics

A total of 122 subjects were enrolled in the study. All 122 subjects were included in the safety and PD analysis sets, and all 94 subjects who received active treatment (Compound IA) were included in the PK analysis set. Overall, 58 (48%) male and 64 (52%) female subjects between 18 and 64 years of age and with a BMI between 18.9 and 29.4 kg/m² participated in the study. The majority of subjects 105 (86%) (Part A, n=57; Part C, n=42 and Part D, n=6) were white.

Of the enrolled subjects, 107 (88%) subjects completed the study as per protocol and 15 (12%) subjects discontinued the study early. These early discontinuations included 1 out of 64 (2%) subjects in Part A, 13 out of 52 (25%) subjects in Part C, and 1 out of 6 (17%) subjects in Part D of the study. Reasons for study discontinuation included withdrawal due to adverse effects (AEs) in 12 (10%) subjects, and 1 (1%) subject each discontinued the study due to either withdrawal of consent, lost to follow-up, or study on temporary hold due to the COVID-19 pandemic (preventing visits; unrelated to the safety of Compound IA). All 4 discontinued subjects were replaced in Part C.

### Safety

Single and multiple doses of Compound IA were generally well tolerated. No deaths or serious adverse events (SAEs) were reported during the study. Overall, 87/122 subjects (71%) reported treatment emergent adverse events (TEAEs); 66/94 subjects (70%) in Compound IA arm, and 21/28 (75%) subjects in placebo arm. The majority of TEAEs reported by 84 (69%) subjects were of mild intensity, whereas 15 subjects (12%) reported moderate TEAEs. The frequently reported system organ class AEs in >20% of the subjects were nervous system disorders (34%), general disorders and administration site conditions (29%), and gastrointestinal disorders (27%).

Collectively, 46 related TEAEs reported by 24/122 subjects (20%) were considered to be related to study drug, including 21/94 (22%) who received Compound IA and 3/28 (11%) subjects who received placebo. For 12/122 (10%) subjects, 20 TEAEs of maculopapular skin rash and/or pruritus were considered adverse events of special interest. All 12 subjects received Compound IA; either as single dose (100 mg [n=1] or 600 mg [n=1]), or multiple dose (30 mg QD [n=2], 100 mg QD [n=3], 200 mg QD [n=2], or 50 mg BID [n=3]). These TEAEs were of mild to moderate intensity, generally started within 1 to 17 days after initiation of treatment with Compound IA and resolved within 1 to 18 days after onset; in all cases without concomitant treatment. For 10 subjects, these TEAEs led to treatment discontinuation. Two other subjects were early discontinued due to TEAEs that were unrelated to the study drug.

Mild decreases in neutrophil and leukocyte counts were considered non-clinically significant and occasionally noted, which could be consistent with a PD effect of COMPOUND IA resulting from inhibition of IL-1β signaling downstream of NLRP3. One subject had a second-degree atrioventricular block that was not considered to be related to the study drug. No other clinically relevant findings were reported for vital signs, 12-lead ECG, 24-hour Holter monitoring, or physical examination.

### Pharmacokinetics

The exposure to single doses of Compound IA increased in a less than dose-proportional manner when Compound IA was administered as crystalline suspension (3-300 mg), but increased dose-proportionally when administered as SDD suspension (100-600 mg). After QD administration of Compound IA dose range 30-200 mg for 2 weeks, only limited drug accumulation of about 1.1 to 1.3-fold was observed in reaching steady state. This is consistent with a mean t_{1/2} ranging from 9.83 to 16.2 hours across QD and BID dose levels. At steady state, Compound IA demonstrated a very low CLss/F (˜0.83 to 1.11 L/h) and Vss/F (˜12.6 to 23.3 L), with low-to-moderate inter-subject variability across QD and BID dose levels of Compound IA. Renal clearance at steady state was relatively low (˜0.008 L/h) compared to total oral clearance, hence unlikely to be a relevant clearance pathway in humans.

Administration of a single dose of 100 mg Compound IA as crystalline suspension under fed conditions led to 2.05-fold increase of Cₘₐₓ and 1.49-fold for AUC₀₋ₗₐₛₜ of Compound IA compared to fasted conditions. For the crystalline tablet (100 mg Compound IA under fasted conditions), the median tₘₐₓ of Compound IA was delayed from 2 to 5 hours, the Cₘₐₓ was 78% lower, and the t_{1/2} was comparable between the crystalline tablet and suspension. The encapsulated crystalline tablets (25 mg and 50 mg bid under fed conditions) were characterized by a median lag time of 0.75 and 0.25 hours, respectively, and a median tₘₐₓ of 4 hours on Day 1. The mean t_{1/2} of Compound IA was comparable between the tablet (18.6 hours) and suspension (17.7 hours) formulations.

### Pharmacodynamics

Dose-dependent decreases in concentrations of IL-1β (with mean nadir concentrations of approximately 5% to 20% of the baseline value) were observed with increasing single and multiple oral doses of Compound IA. At most dose levels of Compound IA, the inhibition of IL-1β was observed from 1 hour after dosing until the last sampling time point for single (Day 3 or up to 6 hours for the lowest≤10 mg dose levels) and multiple (Day 15) oral doses of Compound IA.

Based on the fractional maximum stimulation effect (Eₘₐₓ) model tested with a Hill coefficient, the arithmetic mean (±SD) of the observed stimulation effect of IL-1β was 1820 (±102) ng/L, and the Eₘₐₓ of IL-1β was −0.985 (±0.00277). The median potency of Compound IA inhibiting 90% of the ex-vivo stimulated IL-1β release (IC₉₀) in the (LPS) challenge was a concentration of 3.18 μM (90% Cl: 2.84; 3.54). The effective concentrations relative to the estimated maximum therapeutic effect and inhibitory concentrations relative to 100% inhibition of Compound IA resulting from ex-vivo stimulated IL-1β release were EC₅₀: 0.141 μM (90% Cl: 0.114, 0.171), EC₉₀: 2.57 μM (90% Cl: 2.24, 2.94), and IC₅₀: 0.146 μM (90% Cl: 0.118, 0.179)

### Discussion

Single and multiple doses of Compound IA or placebo were generally well tolerated. No deaths or serious adverse events (SAEs) were reported during the study. TEAEs like skin rash and/or pruritus were considered related to the study drug. The majority of TEAEs reported by subjects were mild (69%) and moderate (12%) in severity. The maculopapular and/or pruritic skin rashes were most frequently reported at the higher multiple dose levels of Compound IA, suggesting a relationship with exposure to Compound IA.

Following single oral doses of Compound IA under fasted conditions, Compound IA was rapidly absorbed with a median tₘₐₓ ranging from 0.76 to 3.00 hours across dose levels. However, with higher dose range 30-600 mg the median tₘₐₓ was slightly delayed (1.5 to 3.0 hours) indicating a slower absorption compared to the lower doses (3 and 10 mg: 0.76 and 1.00 hours, respectively). The increase in drug exposure was less than dose-proportional with crystalline suspension (particularly 100 and 300 mg), whereas dose-proportional increase in exposure was observed with SDD suspension (100-600 mg), indicating solubility-limited absorption of crystalline material at doses≥100 mg.

Multiple doses and formulations of Compound IA showed no deviations from dose proportional drug exposure after 2 weeks signifying that multiple dose PK was linear and were not limited by solubility. Following oral doses of Compound IA on Day 1, a slight delay in absorption was observed with encapsulated crystalline tablets under fed condition. This slower absorption was in line with bioavailability results where no clear effect of food on tₘₐₓ was observed. These findings suggest the lag absorption time was due to encapsulation. Renal clearance was determined to be about 0.004 L/h (Day 1) or 0.008 L/h (Day 14), approximating to less than 0.8% of oral dose. This shows that direct secretion of the parent drug into urine is not expected to be a major elimination route for this drug in humans.

Compound IA as 100 mg crystalline tablet showed a positive food effect with increased Cₘₐₓ and AUC by 2.05 and 1.49-fold in the fed (high-fat, high-calorie meal) vs fasted state, respectively. Median Tₘₐₓ for 100 mg crystalline tablet was 5 hours, while shorter Tₘₐₓ values (0.76-3.0 hours) were reported for suspensions. Compound IA has a very low oral clearance (CLss/F˜1.0 L/h), which relates to ≤2% of human liver blood flow and a low volume of distribution (Vss/F) of ˜12.6-23.3 L. Slight drug accumulation of about 1.2-fold was observed after once daily dosing and 2-fold after twice daily dosing was observed in reaching steady state consistent with an effective half-life of approximately 10 hours as determined for crystalline tablet when given with food.

Nonclinical studies have suggested that Compound IA blocks the release of IL-1β using broad range of NLRP3-dependent activators. This has been observed with di-aryl sulfonylurea compounds which are structurally similar to Compound IA [15]. In this study, dose-dependent decreases in concentrations of IL-1β were observed with increasing single and multiple oral doses of Compound IA. IL-1 B production can be mediated by other inflammasomes or by inflammasome independent pathways; thus, inhibitors aimed at IL-1β can result in unintentional immunosuppressive effects. Therefore, pharmacological inhibitors which specifically target the NLRP3 inflammasome only could be a better option for treatment of NLRP3-associated disease. Safety laboratory findings were mild, non-clinically significant decrease in neutrophil and leukocyte counts in 27 subjects. This may be consistent with a PD effect of Compound IA resulting from inhibition of signaling downstream of NLRP3, similar to known effects of anti-IL-1β monoclonal antibody canakinumab.

The AUCs of subjects with heterogenous CYP2C9 genotypes were higher than those observed in subjects with normal CYP2C9 activity. These results suggest that the clearance of Compound IA is affected by reduced CYP2C9 activity caused by specific genetic variants.

In summary, single and multiple oral doses of Compound IA were well tolerated for up to 14 days in healthy subjects, with no safety or tolerability concerns. The PK profile of Compound IA is compatible with a twice daily dosing regimen. The safety and tolerability, PK, and PD results suggests that Compound IA has the potential to be an effective oral first-in-class innate immune modulator warranting further clinical evaluation.

### Example 3

The following procedures are suitable for testing the activity of NLRP3 inhibitors, as per those disclosed herein.

### Procedure 1: IL-1β Production in PMA-Differentiated THP-1 Cells Stimulated with Gramicidin

THP-1 cells were purchased from the American Type Culture Collection and sub-cultured according to instructions from the supplier. Cells were cultured in complete RPMI 1640 (containing 10% heat inactivated FBS, penicillin (100 units/ml) and streptomycin (100 μg/ml)), and maintained in log phase prior to experimental setup. Prior to the experiment, compounds were dissolved in dimethyl sulfoxide (DMSO) to generate a 30 mM stock. The compound stock was first pre-diluted in DMSO to 3, 0.34, 0.042 and 0.0083 mM intermediate concentrations and subsequently spotted using Echo550 liquid handler into an empty 384-well assay plate to achieve desired final concentration (e.g. 100, 33, 11, 3.7, 1.2, 0.41, 0.14, 0.046, 0.015, 0.0051, 0.0017 μM). DMSO was backfilled in the plate to achieve a final DMSO assay concentration of 0.37%. The plate was then sealed and stored at room temperature until required.

THP-1 cells were treated with PMA (Phorbol 12-myristate 13-acetate) (20 ng/ml) for 16-18 hours. On the day of the experiment the media was removed and adherent cells were detached with trypsin for 5 minutes. Cells were then harvested, washed with complete RPMI 1640, spun down, and resuspended in RPMI 1640 (containing 2% heat inactivated FBS, penicillin (100 units/ml) and streptomycin (100 μg/ml). The cells were plated in the 384-well assay plate containing the spotted compounds at a density of 50,000 cells/well (final assay volume 50 μl). Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). The plates were incubated for 18 h at 4° C. and read using the preset HTRF program (donor emission at 620 nm, acceptor emission at 668 nm) of the SpectraMax i3x spectrophotometer (Molecular Devices, software SoftMax 6). A vehicle only control and a dose titration of CRID3 (100-0.0017 μM) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

### Procedure 2: IL-1β Production in PMA-Differentiated THP-1 Cells Stimulated with Gramicidin

THP-1 cells were purchased from the American Type Culture Collection and sub-cultured according to instructions from the supplier. Prior to experiments, cells were cultured in complete RPMI 1640 (containing 10% heat inactivated FBS, penicillin (100 units/ml) and streptomycin (100 μg/ml)), and maintained in log phase prior to experimental setup. Prior to the experiment THP-1 were treated with PMA (Phorbol 12-myristate 13-acetate) (20 ng/ml) for 16-18 hours. Compounds were dissolved in dimethyl sulfoxide (DMSO) to generate a 30 mM stock. On the day of the experiment the media was removed and adherent cells were detached with trypsin for 5 minutes. Cells were then harvested, washed with complete RPMI 1640, spun down, resuspended in RPMI 1640 (containing 2% heat inactivated FBS, penicillin (100 units/ml) and streptomycin (100 μg/ml). The cells were plated in a 384-well plate at a density of 50,000 cells/well (final assay volume 50 μl). Compounds were first dissolved in assay medium to obtain a 5× top concentration of 500 μM. 10 step dilutions (1:3) were then undertaken in assay medium containing 1.67% DMSO. 5× compound solutions were added to the culture medium to achieve desired final concentration (e.g. 100, 33, 11, 3.7, 1.2, 0.41, 0.14, 0.046, 0.015, 0.0051, 0.0017 μM). Final DMSO concentration was at 0.37%. Cells were incubated with compounds for 1 hour and then stimulated with gramicidin (5 μM) (Enzo) for 2 hours. Plates were then centrifuged at 340 g for 5 min. Cell free supernatant (40 μL) was collected using a 96-channel PlateMaster (Gilson) and the production of IL-1β was evaluated by HTRF (cisbio). A vehicle only control and a dose titration of CRID3 (100-0.0017 M) were run concurrently with each experiment. Data was normalized to vehicle-treated samples (equivalent to 0% inhibition) and CRID3 at 100 μM (equivalent to 100% inhibition). Compounds exhibited a concentration-dependent inhibition of IL-1β production in PMA-differentiated THP-1 cells.

### Procedure 3

### 1. Experimental Procedure:

### 1.1 Cell Culture

1) Culture THP-1 cells in the complete RPMI-1640 medium with 10% FBS at 37° C., 5% CO₂.
2) Passage the cells every 3 days by inoculating 3×10⁵ cells per ml.

### 1.2 Compound Preparation

Prepare the 3-fold serial dilution of the compounds with DMSO in a 384-well LDV Microplate using TECAN EVO system to generate the compound source plate with 10 concentrations. Top concentration is 30 mM.

### 1.3 Cell Preparation

1) Centrifuge THP-1 cells at 350 g for 5 min.
2) Re-suspend cells with complete RMPI-1640 medium, and count cells.
3) Seed cells in T225 flask, about 2.5×107 per flask, treat cells with 20 ng/ml PMA (final DMSO concentration<1%).
4) Incubate overnight.

### 1.4 THP-1 Stimulation

1) Wash adherent THP-1 cells with PBS, and detach cells with 4 ml trypsin for T225 flask.
2) Centrifuge cells at 350 g for 5 min, re-suspend cells with RPMI-1640 containing 2% FBS and count cells with trypan blue.
3) Transfer 50 nl/well the serial dilution of test compound to 384-well plate by Echo; For the high control and first point of CRID3 (MCC950), transfer 165 nl, then backfill to make the DMSO concentration is consistent in all wells, the plate layout is as below.
4) Seed 50 k cells in 40 μl RPMI-1640 with 2% FBS per well in 384-well plate.
5) Incubate for 1 h at 37° C., 5% CO₂.
6) Prepare 5× gramicidin, add 10 μl per well, the final concentration is 5 μM, incubate for 2 hrs at 37° C., 5% CO₂.
7) Centrifuge at 350 g for 1 min.
   Pipet 16 μl supernatant by apricot, and transfer into white 384 proxiplate. HC: 100 μM CRID3 (MCC950)+5 μM gramicidin LC: 5 μM Gramicidin.

### 1.5 IL-1β Detection

1) Homogenize the 5× diluent #5 with a vortex and add 1 volume of stock solution in 4 volumes of distilled water.
2) Thaw 20× stock solution of anti-IL1β-Cryptate-antibody and anti-IL1β XL-antibody. Dilute these two antibodies to 1× with detection buffer #3.
3) Pre-mix the two ready-to-use antibody solutions just prior to use.
4) Dispense 4 μl of pre-mixed Anti-IL1β antibodies working solution into all wells.
5) Seal the plate and incubate overnight at 4° C.
6) Read the cell plate using EnVison and plot Readout vs. the test compound concentration to calculate the IC₅₀.

### 2. Data Analysis:

1. IC₅₀ of compounds can be calculated using the following formulas Formula for IC₅₀

$$\% inhibition = 100 - 100 \times [ {HC}_{a v e} - Readout / ( {HC}_{a v e} - L {C}_{a v e} ) ]$$

2. Fit the normalized data in a dose-response manner using XLfit, and calculate the compound concentration.

The following table shows the biological activity of compounds in hTHP-1 assay containing 2% fetal bovine serum: <0.008 μM=“++++++”; ≥0.008 and <0.04 μM=“+++++”; ≥0.04 and <0.2 μM=“++++”; ≥0.2 and <1 μM=“+++”; ≥1 and <5 μM=“++”; ≥5 and <30 μM=“+”.

| Compound | hTHP-1 IC₅₀ |
|---|---|
| I | ++++ |
| IA | +++++ |
| IB | +++ |

All publications and patent documents cited herein are incorporated herein by reference as if each such publication or document was specifically and individually indicated to be incorporated herein by reference. The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the invention that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

1. A method of treating osteoarthritis, the method comprising administering an NLRP3 inhibitor, wherein the NLRP3 inhibitor is administered to a subject at a total daily dose of about 10 mg to about 100 mg in a single dose or divided doses.

2. The method according to claim 1, wherein the NLRP3 inhibitor is administered to a subject at a total daily dose of about 20 mg to about 50 mg in a single dose or divided doses.

3. The method according to claim 1, wherein the NLRP3 inhibitor is administered to a subject at a total daily dose of about 20 mg in a single dose or divided doses.

4. The method according to claim 1, wherein the NLRP3 inhibitor is administered to a subject at a total daily dose of about 50 mg in a single dose or divided doses.

5. The method according to claim 1, wherein the NLRP3 inhibitor is administered to a subject at a dose of about 10 mg twice daily.

6. The method according to claim 1, wherein the NLRP3 inhibitor is administered to a subject at a dose of about 10 mg twice daily for about 14 consecutive days.

7. The method according to claim 1, wherein the NLRP3 inhibitor is administered to a subject at a dose of about 25 mg twice daily.

8. The method according to claim 1, wherein the NLRP3 inhibitor is administered to a human subject at a dose of about 25 mg twice daily for about 70 consecutive days.

9. The method according to claim 1, wherein the NLRP3 inhibitor is administered to a subject during or after consuming food.

10. The method according to claim 1, wherein there is about a 10-14 hour time interval between the administration of two subsequent doses of the NLRP3 inhibitor to a subject.

11. The method according to claim 1, wherein said osteoarthritis is knee osteoarthritis.

12. The method according to claim 1, wherein administration of the NLRP3 inhibitor decreases pain in the osteoarthritis affected joint as determined by KOOS score based on change from baseline.

13. The method according to claim 1, wherein administration of the NLRP3 inhibitor reduces the inflammation level of the osteoarthritis affected joint as determined by change from baseline in synovitis activity level measured from K^{trans} by dynamic contrast-enhanced (DCE)-MRI.

14. The method according to claim 1, wherein the level of serum high sensitivity C-Reactive Protein decreases in a subject by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% as determined by change from baseline.

15. The method according to claim 1, wherein the level of IL-1β or IL-18 decreases in a subject by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% as determined by change from baseline.

16. The method according to claim 1, wherein the subject does not exhibit any skin rash.

17. The method according to claim 1, wherein the NLRP3 inhibitor is administered to the subject orally.

18. The method according to claim 1, wherein the NLRP3 inhibitor is comprised in a tablet formulation.

19. The method according to claim 1, comprising administering at least one further therapeutic agent.

20. The method according to claim 1, wherein the NLRP3 inhibitor is Compound I, or a pharmaceutically acceptable salt thereof: 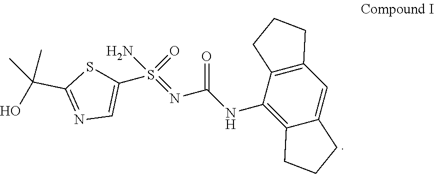 [SMILES: CC(C)(O)c1ncc(S(N)(=O)=NC(=O)Nc2c3c(cc4c2CCC4)CCC3)s1]
